(19) United States
(12) Patent Application Publication
(10) Pub. No.: US 2024/0091410 A1
(43) Pub. Date: Mar. 21, 2024

(54) Vaginal Encapsulation Devices

(71) Applicant: OAK CREST INSTITUTE OF SCIENCE, Monrovia, CA (US)

(72) Inventors: Marc M. Baum, Altadena, CA (US); Peter A. Anton, Monrovia, CA (US); Manjula Gunawardana, Claremont, CA (US); John A. Moss, Sierra Madre, CA (US)

(21) Appl. No.: 18/038,234

(22) PCT Filed: Nov. 24, 2021
(86) PCT No.: PCT/US21/60815

§ 371 Date: May 23, 2023

Related U.S. Application Data
(60) Provisional application No. 63/118,622, filed on Nov. 25, 2020.

(51) Int. Cl. A61L 27/36; A61L 27/16; A61L 27/52; A61L 27/54; A61L 27/58

(52) U.S. Cl. CPC A61L 27/3679; A61L 27/16; A61L 27/52; A61L 27/54; A61L 27/58; A61L 2300/256; A61L 2300/64

(57) ABSTRACT

Provided herein are drug delivery devices, comprising a scaffold comprising one or more biocompatible materials, one or more chambers containing a plurality of cells, one or more membranes, and one or more nutrient supplementation systems. Also provided are methods of treating or preventing diseases and disorders in a subject in need thereof, comprising administering to the subject the drug delivery devices disclosed herein, and methods of making the drug delivery devices disclosed herein.

### CROSS-REFERENCE TO RELATED APPLICATIONS

This application claims priority to U.S. Ser. No. 63/118,622, filed Nov. 25, 2020, the disclosure of which is hereby incorporated by reference in its entirety.

### FIELD OF INVENTION

This disclosure is generally in the field of cellular encapsulation devices for vaginal use.

### BACKGROUND

The use of foreign live organisms to produce biologically active compounds in vivo (i.e., in an anatomic compartment of a living human host or other mammal) as a strategy for the prevention or treatment of disease is appealing based on considerations relating to cost and logistics. Ideally, a mucosal surface such as the buccal, nasal, or vaginal mucosa would be inoculated at repeated intervals with the organism to establish and maintain a community that produces the biologically active agent(s). This strategy is being exploited in two main areas: delivery of prophylactic and therapeutic active pharmaceutical ingredients (APIs), and delivery of agents that elicit an immune response in the host, possibly leading to immunization against a disease. Illustrative examples of both strategies in the context of delivery to the vaginal mucosa are provided below.

### SUMMARY

Provided herein are drug delivery devices, comprising a scaffold comprising one or more biocompatible materials, one or more chambers containing a plurality of cells, one or more membranes, and one or more nutrient supplementation systems. Particularly provided are drug delivery devices which are adapted for intravaginal use. In some embodiments, the plurality of cells comprises bacterial cells, fungal cells, mammalian cells, or a combination thereof. In some embodiments, the plurality of cells comprises bacterial cells, e.g., one or more members of the Lactobacillus genus.

Also provided are methods of treating or preventing diseases and disorders in a subject in need thereof, comprising administering to the subject a drug delivery device disclosed herein. In some embodiments, the disease or disorder is a sexually transmitted infection (STI).

Further provided are methods of contraception, comprising administering to a subject in need thereof a drug delivery device disclosed herein.

### BRIEF DESCRIPTION OF THE DRAWINGS

FIG. 1 shows exemplary embodiments of intravaginal drug delivery device designs.

FIG. 2 shows an alternative exemplary embodiment of an intravaginal drug delivery device design with a cylindrical chamber/membrane inside a perforated scaffold.

FIG. 3 shows an alternative exemplary embodiment of an intravaginal drug delivery device design with discrete chambers in a non-toroidal geometry.

FIG. 4 shows an alternative exemplary embodiment of an intravaginal drug delivery device design in a cassette shape and with a mesh outside the membrane covering the chamber.

FIG. 5 shows an alternative exemplary embodiment of an oblong intravaginal drug delivery device design in a cassette shape with a mesh outside the membrane covering the chamber.

FIG. 6 shows an alternative embodiment of an intravaginal drug delivery device having an upper and lower portion of a scaffold defining a chamber.

FIG. 7 shows an alternative embodiment of an intravaginal implant 700, having a cutout, 701, located in a lobe that protrudes inward from the outer edge of the drug delivery device, and where the cutout holds a capsule-shaped drug delivery device disclosed herein containing cells.

FIG. 8 shows an alternative embodiment of an intravaginal implant similar to 700, but with two or more lobes containing two or more cell capsule-shaped drug delivery devices disclosed herein, 800.

FIG. 9 shows an embodiment of a capsule-shaped drug delivery device for intravaginal implants shown in FIG. 7 and FIG. 8 having a single-membrane with a sealing disk to enclose the drug delivery device.

FIG. 10 shows an alternative embodiment of a capsule-shaped drug delivery device for intravaginal implants shown in FIG. 7 and FIG. 8 where the body serves as the cell chamber and is sealed by a disk comprising a membrane, 1010.

FIG. 11 shows an alternative embodiment of an intravaginal drug delivery device in the shape of a capsule and having a dual-membrane design.

FIG. 12 shows an alternative embodiment of a disk design for a capsule-shaped drug delivery device.

FIG. 13 shows exemplary embodiments of pessary intravaginal drug delivery device designs.

FIG. 14 shows exemplary embodiments of intrauterine device (IUD) designs.

FIG. 15 is a graph illustrating release of luciferase over time in connection with cultivation of “free” THP-1-Dual monocytes (i.e., not encapsulated). The cells produce fluorescent luciferase in the presence of the inducer (circles), but not when the inducer is omitted from the growth medium (squares). Each timepoint corresponds to the mean±SD of three biological replicates. Arrows identify media exchanges that include a fresh supply of inducer.

FIG. 16 is a graph illustrating release of luciferase over time in connection with cultivation of encapsulated THP-1-Dual monocytes. The cells produce fluorescent luciferase in the presence of the inducer and the release rate is linear; 77 ng d⁻¹, R²=0.9077. Each timepoint corresponds to the mean±SD of three biological replicates. Arrows identify media exchanges that include a fresh supply of inducer.

### DETAILED DESCRIPTION OF THE INVENTION

The present disclosure takes advantage of the benefits of live organisms in preventing and treating disease while overcoming inherent disadvantages associated with the technique in a manner that has not been reported previously. Various components, including cells, can be encapsulated within a vaginal device comprising a semipermeable membrane and one or more reservoir chambers. The semipermeable membrane typically permits access of nutrients, growth factors, and small biological agents to the encapsulated cells, but prevents access of cells of the immune system. The semipermeable membrane also can prevent egress of the cells from the encapsulation device, which can be safely removed in its entirety.

This disclosure provides drug delivery devices, e.g., vaginal encapsulation devices, comprising a scaffold comprising one or more biocompatible materials, one or more chambers containing a plurality of cells, one or more membranes, and one or more nutrient supplementation systems. The device prevents the release of the cells, but is permeable to one or more biologically active agents produced by the cells. A variety of device configurations and uses are described as well as methods of manufacture thereof. The device is biocompatible and biostable, and is useful in patients—both humans and animals—for the delivery of appropriate bioactive substances (e.g., to the vaginal mucosa). Also provided are methods of using the disclosed drug delivery devices, e.g., for treating or preventing diseases and disorders, such as sexually transmitted infections (STIs) and pregnancy.

All references cited herein are incorporated by reference in their entirety as though fully set forth. Unless defined otherwise, technical and scientific terms used herein have the same meaning as commonly understood by one of ordinary skill in the art. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (Boca Raton, FL, 2008); Oxford Textbook of Medicine, Oxford Univ. Press (Oxford, England, UK, May 2010, with 2018 update); Harrison's Principles of Internal Medicine, Vol. 1 and 2, 20^{th} ed., McGraw-Hill (New York, NY, 2018); Singleton and Sainsbury, Dictionary of Microbiology and Molecular Biology, 3^{rd} ed., revised ed., J. Wiley & Sons (New York, NY, 2006); Smith, March's Advanced Organic Chemistry Reactions, Mechanisms and Structure 7^{th} ed., J. Wiley & Sons (New York, NY, 2013); and Singleton, Dictionary of DNA and Genome Technology, 3_{rd} ed., Wiley-Blackwell (Hoboken, N J, 2012), provide one skilled in the art with a general guide to many of the terms used in the present application.

One skilled in the art will recognize many methods and materials similar or equivalent to those described herein, which could be used in the practice of the present disclosure. Indeed, the present disclosure is in no way limited to the methods and materials described. For purposes of the present disclosure, certain terms are defined below.

The disclosure provides materials and methods for treating or preventing “conditions” and “disease conditions,” which include, but are not limited to, treating, preventing, reducing the likelihood of having, reducing the severity of, and/or slowing the progression of a medical condition in a subject, also termed “application” hereunder. Such conditions or applications can be remedied through the use of one or more agents administered through a cellular encapsulation device (e.g., vaginal cellular encapsulation device).

These conditions, or applications, are described further under “Use and Applications of the Device” and may include, but are not limited to, infectious diseases (e.g., a human immunodeficiency virus (HIV) infection, acquired immune deficiency syndrome (AIDS), a herpes simplex virus (HSV) infection, a hepatitis virus infection, an influenza infection, tuberculosis, other bacterial infections, and malaria), microbial dysbiosis (e.g., bacterial vaginosis), diabetes, cardiovascular disorders, cancers, autoimmune diseases, central nervous system (CNS) conditions, and analogous conditions in non-human mammals.

In addition, the disclosure includes the administration of biologics produced by the encapsulated organisms, such as proteins and peptides, for the treatment or prevention of a variety of disorders such as, for instance, conditions treatable with leuprolide (e.g., anemia caused by bleeding from uterine leiomyomas, fibroid tumors in the uterus, cancer of the prostate, and central precocious puberty), exenatide for the treatment of diabetes, histrelin acetate for the treatment for central precocious puberty, etc. A more detailed list of illustrative examples of potential applications of the disclosure is provided under “Use and Applications of the Device”.

As used herein, the term “HIV” includes HIV-1 and HIV-2.

As used herein, the term “agent” refers to a pharmaceutically active substance produced by cells disclosed herein, including without limitation any cell-produced molecules, ions, polymers, and particles that possess a desirable biological activity.

As used herein, the term “drug”, “medicament”, and “therapeutic agent” are used interchangeably.

As used herein, the term “API” means active pharmaceutical ingredient, which includes agents described herein.

One of ordinary skill will appreciate that any disclosure herein relating to “drug delivery system” also applies to “vaginal encapsulation device” and vice versa, unless otherwise indicated.

The term “IVR” means intravaginal ring.

The term “IUD” means intrauterine device.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult, children, and newborn subjects, whether male or female, are intended to be included within the scope of this term.

With the foregoing in mind, in various embodiments, the disclosure provides devices, systems and methods for treating, preventing, reducing the likelihood of having, reducing the severity of and/or slowing the progression of a condition in a subject.

### Drug Delivery Devices (e.g., Vaginal Cell Encapsulation Devices)

The drug delivery devices disclosed herein comprise the following fundamental components:

A scaffold comprising one or more biocompatible materials; one or more chambers containing a plurality of cells; a membrane; and one or more nutrient supplementation systems.

The drug delivery devices can optionally comprise one or more additional chambers or compartments. Particularly disclosed are drug delivery devices adapted for intravaginal use, i.e., vaginal encapsulation devices. Additional details on exemplary embodiments are provided below.

### Encapsulation System Architecture

A wide variety of architectures are possible in the context of the instant disclosure, as described in detail under “Encapsulation Device Types” below.

### Drug Delivery Device Scaffold

The drug delivery devices disclosed herein comprise a scaffold comprising one or more biocompatible materials. The scaffold has various functions for the device, as described below. In one embodiment, the device comprises a scaffold on which one or more membranes (e.g., semipermeable layers) are mounted. The scaffold provides support to the membranes and positions them so as to define the dimensions of the chamber. The scaffold surrounds a perimeter of the one or more membranes, e.g., such that it closes off the space between the membranes at the periphery of the chamber.

In some embodiments, the scaffold is chemically inert. In some embodiments, the scaffold provides stable mechanical properties to the device. The scaffold can also function to maintain the integrity of the device, e.g., by preventing leakage of cells into or out of the device at the perimeter of the membranes. The scaffold may comprise one or more loading ports to facilitate addition and/or removal of substances to/from the chamber. Optionally, the scaffold (and/or membranes) can be sterilized. In some embodiments, e.g., where the drug delivery device is for vaginal use, the scaffold and/or membranes is not sterilized.

The scaffold may be made from any suitable biocompatible material, preferably durable material that is capable of providing mechanical support to one or more membranes and maintaining the integrity of the device as a whole. The scaffold may, for example, be formed from suitable polymer materials such as silicone, polypropylene or polyetheretherketone (PEEK), or from a ceramic or metallic material such as titanium, titanium alloy or stainless steel, or other suitable materials as described herein (e.g., in the section “Encapsulation Device Scaffold Materials” below).

In embodiments of the present disclosure, the scaffold comprises a first frame element and a second frame element. The first and second frame elements may, for example, engage or interlock mechanically and/or may be welded together to form the overall structure of the scaffold. Ultrasonic welding is used in some embodiments (see “Encapsulation System Fabrication”) to join and/or seal the first and second frame elements. In one embodiment, the first and second frame elements are welded together substantially around the entire perimeter of the scaffold.

In some embodiments, the first and second frame elements function together to position the membranes at a predetermined separation distance between the layers, thereby defining the depth of the chamber. For instance, a first membrane may be mounted on a first frame element and a second membrane on a second frame element. When the first and second frame elements interconnect, they may enclose the perimeter of the chamber. This type of arrangement avoids the need for a separate spacer element, simplifies the manufacture of the device and prevents the exposure of rough edges of the membranes to the exterior of the device. Thus, in various embodiments of the present disclosure, the external profile of the scaffold and the drug delivery device as a whole is as smooth as possible. Without wishing to be bound by any particular theory, this improves biocompatibility and thereby the viability of encapsulated cells following implantation in vivo.

The scaffold may be of any suitable geometry or size within the confines described herein. The scaffold, together with the one or more membranes which are mounted thereon, may define the geometry of the chamber of the device. The chamber may form various shapes or conformations, as shown schematically herein for various embodiments.

In particular embodiments, the separation distance between a first and second membrane (i.e., the depth of the chamber) may be, for example, 0.05 to 10 mm, 0.2 to 5 mm, 0.3 to 1 mm or 0.4 to 0.8 mm. Advantageously, embodiments of the present disclosure allow the first and second membranes to be positioned at a precise separation distance. By controlling the depth of the chamber in this manner, optimal conditions for differentiation, growth and maintenance of the encapsulated cells can be achieved, according to the precise nature of the cells within the chamber. For instance, without wishing to be bound by any particular theory, the separation distance of the membranes can affect the availability of oxygen and other nutrients to cells encapsulated within the chamber, since these materials need to diffuse across the semipermeable layers. Thus, cell types which are growing and/or metabolizing at a higher rate (e.g., that have higher oxygen requirements) may need to be encapsulated in a device with a smaller separation distance between the membranes, compared to cell types having lower metabolic requirements. Embodiments of the present disclosure allow the separation distance to be varied and tested for different cell types, and a suitable depth of the chamber selected for optimal viability of the encapsulated cells.

### Encapsulation Device Scaffold Materials

The drug delivery devices disclosed herein comprise a scaffold comprising one or more biocompatible materials. In various embodiments, one or more biocompatible materials are non-resorbable, e.g., they are substantially inert under physiological conditions and do not substantially biodegrade in the body during the period of use. In various embodiments, one or more biocompatible materials comprise one or more thermoplastic polymers, one or more elastomers, one or more biocompatible metals, or combinations thereof. Non-limiting examples of such materials are described in the literature (1, 2), incorporated by reference in their entirety.

In various embodiments, the biocompatible material is non-resorbable. In various embodiments, the biocompatible material is a non-resorbable elastomer. Non-limiting examples of elastomers include a medical-grade poly(dimethyl siloxane) and silicone. Other non-limiting examples of suitable non-resorbable biocompatible materials include: synthetic polymers selected from poly(ethers); poly(acrylates); poly(methacrylates); poly(vinyl pyrolidones); poly(vinyl acetates), including but not limited to poly(ethylene-co-vinyl acetate) (EVA); poly(urethanes); celluloses; cellulose acetates; poly(siloxanes); poly(ethylene); poly(tetrafluoroethylene) and other fluorinated polymers; poly(siloxanes); copolymers thereof, and combinations thereof. Further non-limiting examples of non-resorbable materials include biocompatible metals such as titanium, stainless steel, and others known to those skilled in the art, or combinations thereof. The term “metals” as used herein includes pure metals and metal alloys.

In various embodiments, the biocompatible material is resorbable. In various embodiments, the biocompatible material is a resorbable elastomer. Non-limiting examples of suitable resorbable biocompatible materials include: synthetic polymers selected from poly(amides); poly(esters); poly(ester amides); poly(anhydrides); poly(orthoesters); polyphosphazenes; pseudo poly(amino acids); poly(glycerol-sebacate); copolymers thereof, and mixtures thereof. In one embodiment, the resorbable synthetic polymers are selected from poly(lactic acids), poly(glycolic acids), poly(lactic-co-glycolic acids), poly(caprolactones) (PCLs), and mixtures thereof. Other curable bioresorbable elastomers include PCL derivatives, amino alcohol-based poly(ester amides) (PEA), poly(octane-diol citrate) (POC), and combinations thereof. PCL-based polymers may require additional cross-linking agents such as lysine diisocyanate or 2,2-bis(-caprolacton-4-yl)propane to obtain elastomeric properties.

In various embodiments of the drug delivery devices described herein, the biocompatible materials comprise any suitable thermoplastic polymers or elastomers suitable for pharmaceutical use, such as silicone, low density polyethylene, EVA, polyurethanes, styrene-butadiene-styrene copolymers, and combinations thereof. In some embodiments, the one or more biocompatible materials comprise silicone, polyurethane, poly(ethylene-co-vinyl acetate) (EVA), or a combination thereof. In some embodiments, the one or more biocompatible materials comprise silicone. In some embodiments, the one or more biocompatible materials comprise polyurethane. In some embodiments, the one or more biocompatible materials comprise poly(ethylene-co-vinyl acetate) (EVA).

### Encapsulation Device Types

Embodiments of the present disclosure relate to drug delivery devices for vaginal use holding living cells, i.e., vaginal encapsulation devices. The embodiments of the drug delivery devices described herein are not intended to be limited to certain device size, shape, design, volume capacity, and/or materials used to make the drug delivery devices.

The disclosure provides drug delivery devices adapted for intravaginal use, i.e., intravaginal devices (including vaginal encapsulation devices). Non-limiting examples of intravaginal devices disclosed herein include intravaginal rings (IVRs), intrauterine devices (IUDs), pessaries, and other types of devices placed in the vagina, such as tampon-shaped rods and the like. In some embodiments, the drug delivery device is an intravaginal ring (IVR), an intrauterine device (IUD), or a pessary. In some embodiments, the drug delivery device is an intravaginal ring (IVR). In some embodiments, the drug delivery device is an intrauterine device (IUD). In some embodiments, the drug delivery device is a pessary.

Drug delivery devices disclosed herein, such as intravaginal devices, contain one or more chambers. In an embodiment, the one or more chambers of the drug delivery device, such as an intravaginal device, contain live cells. In an embodiment, the one or more chambers contain bacterial cells, yeast cells, plant cells, or cells from any eukaryotic multicellular organism, including mammals. The cells are contained in a chamber that is separated from the environment by at least one membrane, and the membrane prevents the release of live cells to the environment, e.g., to the body environment (including the vaginal environment), while being permeable to certain molecules, ions, polymers, or particles produced by the cells. Collectively, said cell-produced molecules, ions, polymers, and particles that possess a desirable biological activity are referred to herein as “agents”. The membrane's primary role is to prevent the release of cells from the device and its purpose is not intended to significantly modify or control the release of the agents from the cell-containing chamber. The rate of release of agents from the device therefore is driven by the rate at which they are released by the cells. In other words, the devices disclosed herein would not be considered a sustained release drug delivery system such as IVRs known in the art (3-9), incorporated herein by reference, where the biomedical materials are engineered to control and sustain the rate of release of the agent for extended periods of time. The release kinetics of the agent(s) from sustained release drug delivery systems (SRDDSs) is controlled by osmosis and/or by diffusion, and is usually zero order, pseudo-zero order, or first order. By contrast, the release of agent(s) from the encapsulated cells of the drug delivery devices disclosed herein is controlled by numerous, complex factors, such as the concentration or nutrients, the metabolic state of the of the cells, the expression of intracellular enzymes and proteins, including transporter proteins, and the permeability (active and/or passive) of the agent(s) across one or more biological membranes. Importantly, the agent(s) is(are) produced by the living cells over time, while a SRDDS contains the entire API payload at administration.

Because the devices disclosed herein are not a SRDDS, embodiments are designed to release agent(s) over periods of time spanning hours to days to weeks to months to years, depending on the target application. In some embodiments, the drug delivery device is physically stable at about 0-50° C. In some embodiments, the drug delivery device is physically stable at about 30-40° C. In some embodiments, the drug delivery device is physically stable at about 37° C.

### Intravaginal Rings

Nonlimiting embodiments of drug delivery devices, such as a vaginal encapsulation device based on an IVR geometry, are described herein with supporting drawings. In one, non-limiting embodiment, drug delivery devices for vaginal use, such as IVRs, are toroidal in geometry (ring-shaped), 104, optionally with an outer diameter of 40-70 mm and a cross-sectional diameter of 2-10 mm. Preferred IVR outer diameters are 50-60 mm, or 54-56 mm and cross-sectional diameters of 3-8 mm, or 4-6 mm. A cross-sectional view, 104a, shows a toroidal geometry device with an outer membrane, 104b, encapsulating a core containing cells and other components such as nutrients, 104c. The cross-sectional shape of IVRs can be other than circular, such as square, rectangular, triangular, or other shapes, 105. The IVR may contain discrete chambers containing cells and other components of the drug delivery function connected by sections of the biocompatible material of the drug delivery device, e.g., an elastomer, that serve to hold the compartments in a ring-like orientation and enable retention of the IVR in the vagina, 106. In another embodiment, a central chamber contains the cells of the drug delivery device, with an outer ring that functions only to retain the device in the vaginal cavity, 107. The drug delivery functionality may be contained in a module that is inserted into a central chamber through an opening, 107a, with multiple large openings allowing the agent(s) produced by the cells to exit the central chamber, but not playing a role in control of the drug's release rate. In an alternate embodiment, both the ring and central chamber may contain drug delivery components.

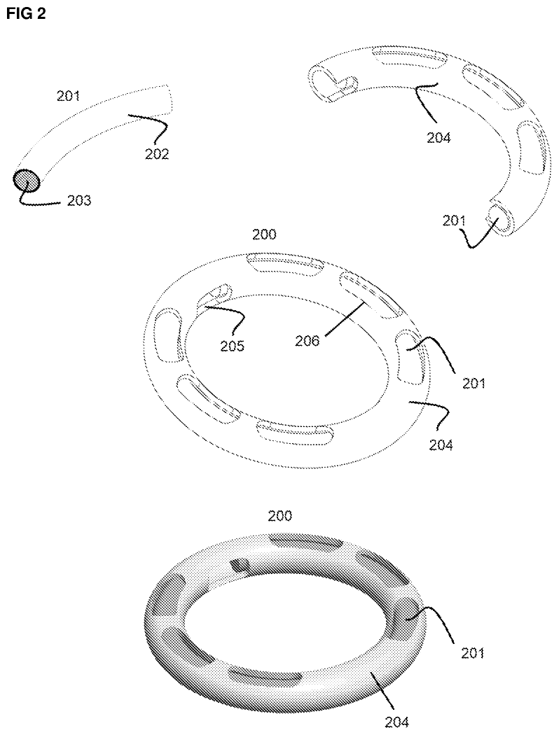

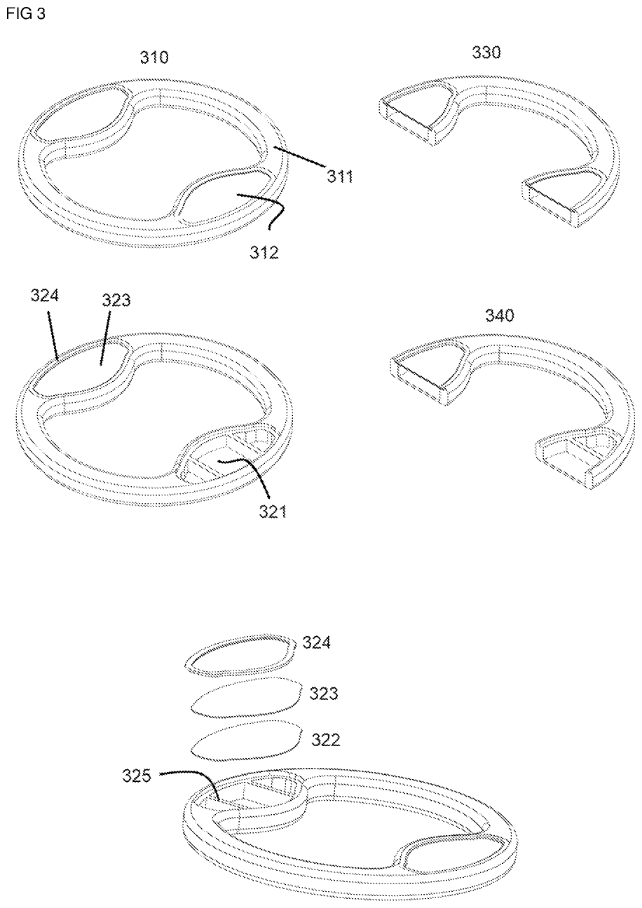

In one embodiment, 200 (FIG. 2), one or more cylindrical chambers, 201, comprising a permeable membrane, 202, enclosed with end-caps and containing a cell-containing core, 203, are held within a perforated skin, 204. In some cases, the skin comprises an elastomer, e.g., a non-medicated elastomer. Core elements are inserted into the drug delivery device through perforations, 205. Additional perforations, 206, in the skin allow the chamber to interact with the vaginal fluids, but perforations do not play a significant role in controlling the agent's release rate. Another embodiment, 310 (FIG. 3), comprises a body structure, 311, with one or more delivery modules, 312, to contain cells with or without excipients. Modules comprise a chamber, 321, enclosed with a discrete, permeable membrane, 322, that is sealed to the drug delivery device body and retains cells within the chamber. An additional protective mesh, 323, may optionally be present on top of the membrane to protect it from puncture. A sealing ring or other structure, 324, may optionally be used to hold the membrane and mesh in place on top of the chamber containing the cells. Chambers may contain ribs, 325, to further subdivide the chambers covered by one membrane structure and to provide support to the membrane and mesh.

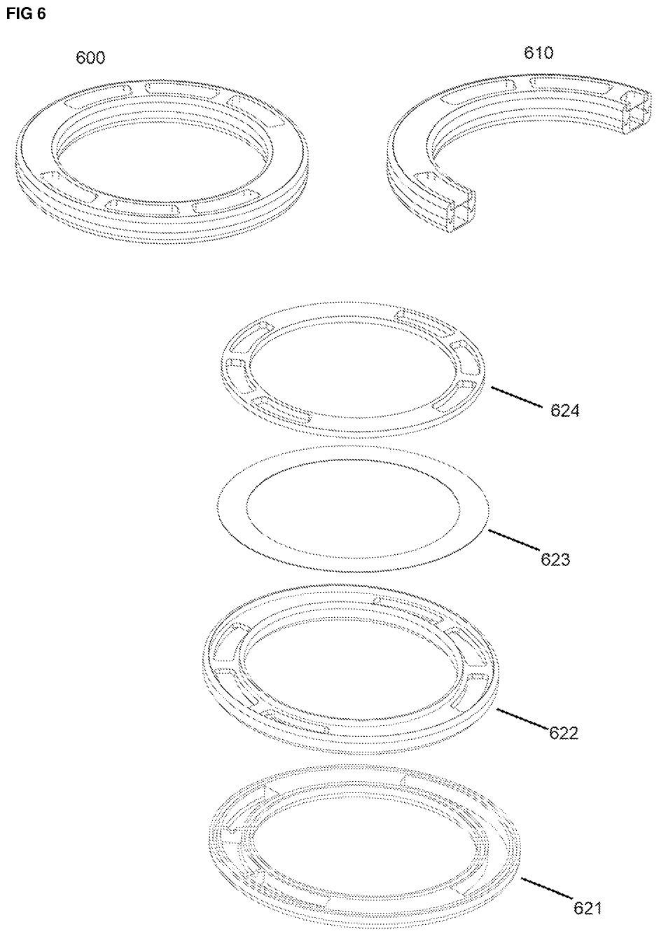

An alternative embodiment, 600 (FIG. 6), also shown in cross-section, 610, comprises a lower element, 621, comprising one or more chambers to contain cells. The chambers are covered by an upper element, 622 that forms the upper portion of the chambers and holds a permeable membrane, 623, on top to retain cells in the chamber. The membrane may be retained in 622 by a ring, 624.

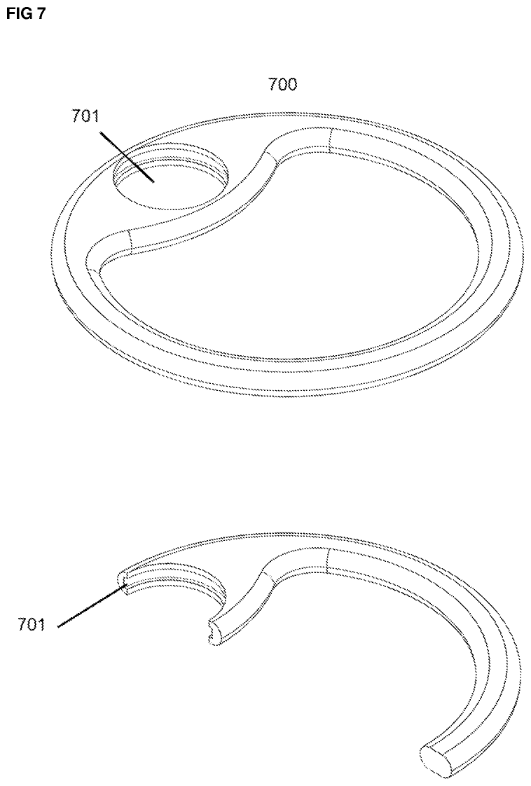

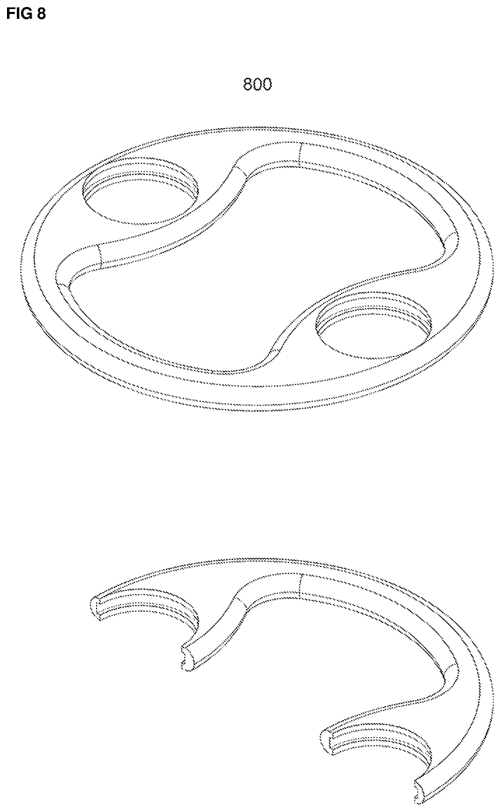

Alternative embodiments employ an elastomer IVR scaffold to hold a chamber, as described below in the section “Other Intravaginal Devices, Including Rods”, and to position the drug delivery device appropriately in the vaginal cavity (FIG. 7). In one embodiment, 700, a cut-out, 701, located in a lobe that protrudes inward from the outer edge of the IVR torus holds the chamber. An alternate embodiment (FIG. 8) has two or more lobes containing two or more chambers, 800.

### Pessaries

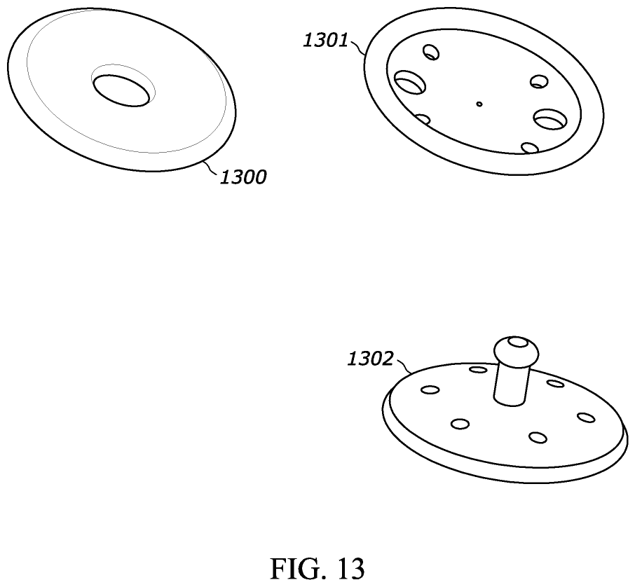

Nonlimiting embodiments of a drug delivery device, e.g., a vaginal encapsulation device, based on a pessary geometry are described herein with supporting drawings in FIG. 13. The figure shows three common pessary geometries that may be employed to hold chambers as described above for embodiments 700 and 800.

### Intrauterine Devices

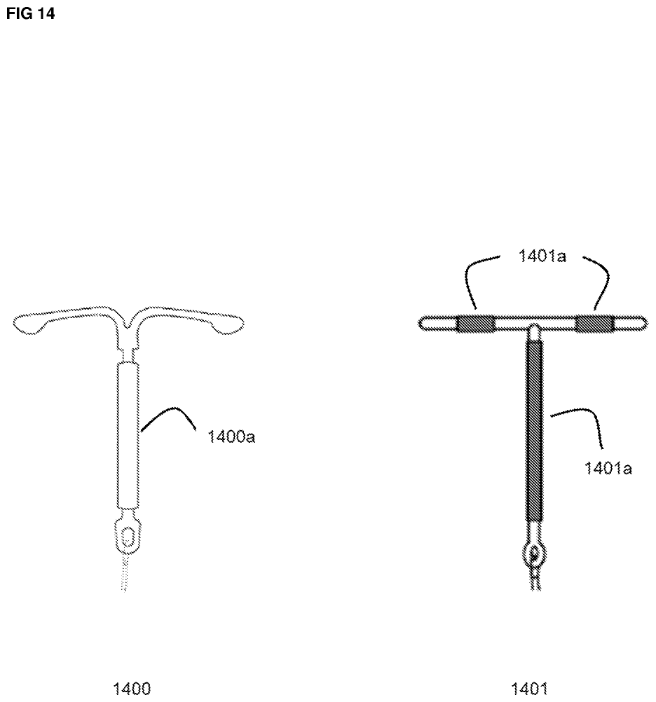

Nonlimiting embodiments of a drug delivery device, i.e, a vaginal encapsulation device, based on an IUD geometry are described herein with supporting drawings in FIG. 14. In one embodiment, 1400, a cylindrical chamber forms the body of an IUD. The membrane, 1400a, retains cells in this chamber. In an alternate embodiment, 1401, cylindrical chambers, 1401a, may comprise both the arms and main body of the IUD.

### Other Intravaginal Devices, Including Rods

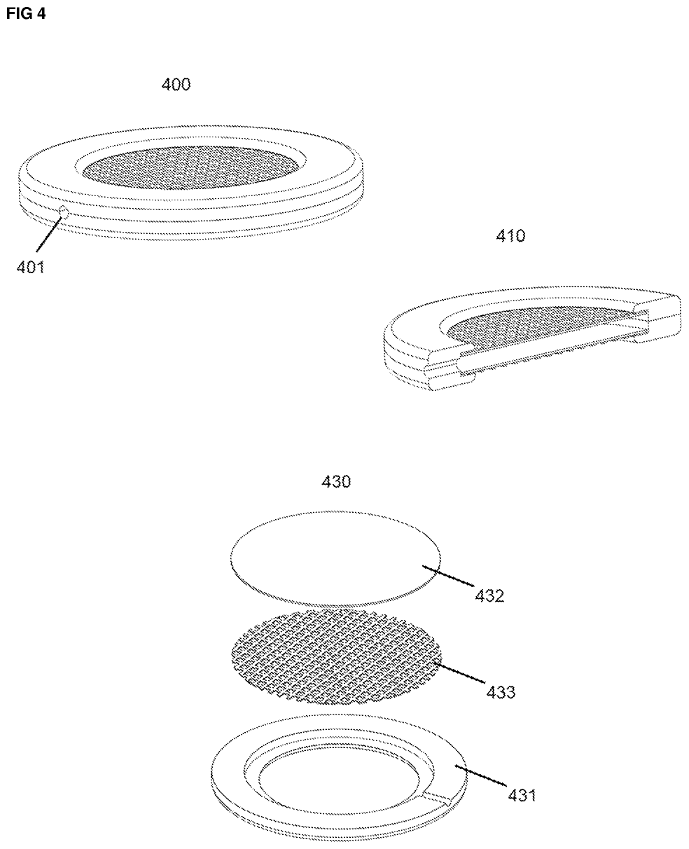

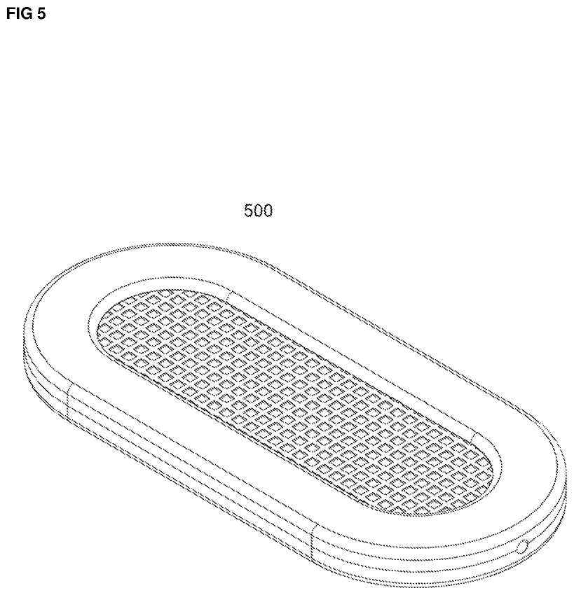

Nonlimiting embodiments of a drug delivery device, e.g., a vaginal encapsulation device, based on other geometries are described herein with supporting drawings. These devices are designed to be inserted into a scaffold (e.g., an elastomer scaffold) such as 700 of FIG. 7, 800 of FIG. 8, or in a pessary-like device such as 1300, 1301, and 1302 in FIG. 13. Retention of the drug delivery device in the vaginal cavity is determined by the geometry of the scaffold and not the chamber that is inserted into the scaffold. In one embodiment, 400 (FIG. 4) a membrane, 432, is inserted into a chamber body structure, 431, with an optional mesh support, 433, placed between the body and the membrane. This forms one half of a chamber 400, with the chamber to hold cells defined by the volume between two membranes and the body walls. A port, 401, may be included to allow filling the chamber with cells and excipients after chamber assembly. The chamber may be of circular geometry, as in 400, oblong geometry, as in 500 (FIG. 5), or any other suitable geometry for insertion into the scaffold of a drug delivery device disclosed here, e.g., a vaginal delivery device.

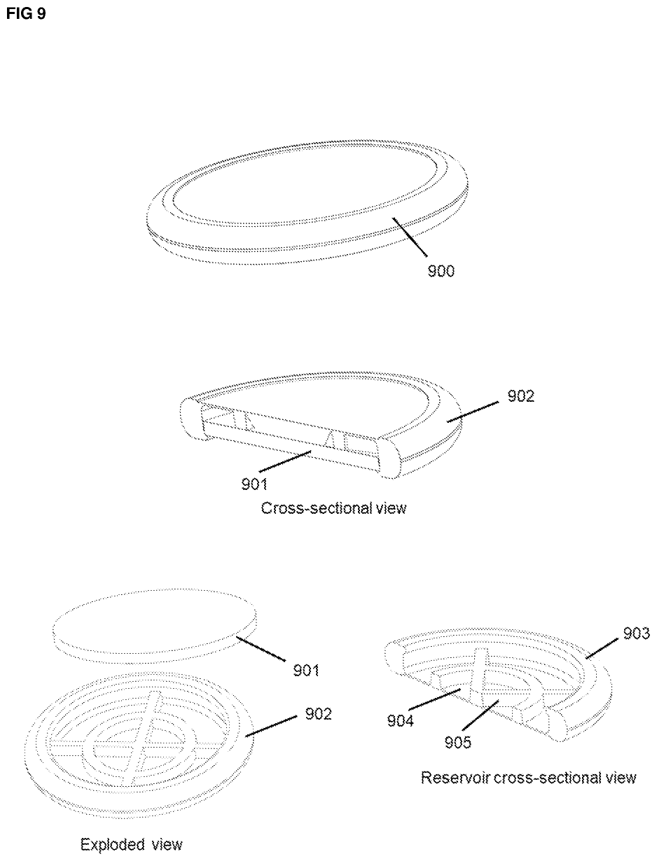

In another embodiment, the drug delivery device is shaped like a capsule, optionally from about 3 to about 50 mm in diameter and up to about 5 mm in height. In some embodiments, e.g., 900 in FIG. 9, the device comprises or consists of a chamber, 902, comprising a scaffold ring (e.g., the scaffold comprising an elastomer) with a circular membrane bonded to it. A non-permeable cover, 901 seals the chamber on the side opposite the membrane. In some embodiments, the chamber comprises an outer sealing ring, 903, that forms a seal with the membrane, 904, and none or one or more rib structures, 905, that support the membrane and define compartments within the chamber. The chamber may be fabricated as a single part from one material, or it may be assembled from a first part comprising the outer sealing ring and any rib structures and a second part comprising a separate membrane that is attached to the first part using adhesive or another assembly method disclosed herein. In any of the embodiments described herein, cells as described herein can be contained in these chambers formed between the inner scaffold surfaces, membrane, and cover. In any of the embodiments described herein, all chambers defined by the rib structures may be filled with cells and suitable excipients, or some chambers may be filled and some remain unfilled. In any of the embodiments described herein, all chambers contain the same cell type. In any of the embodiments described herein different chambers may contain different cell types. In any of the embodiments described herein, the plurality of chambers contains a total of two cell types. In another preferred embodiment, the plurality of chambers contains a total of three or more cell types. Those skilled in the art will recognize from the disclosure provided herein that the compartments in a chamber may contain any of a number of possible combinations of cell types, and all possible combinations are incorporated herein.

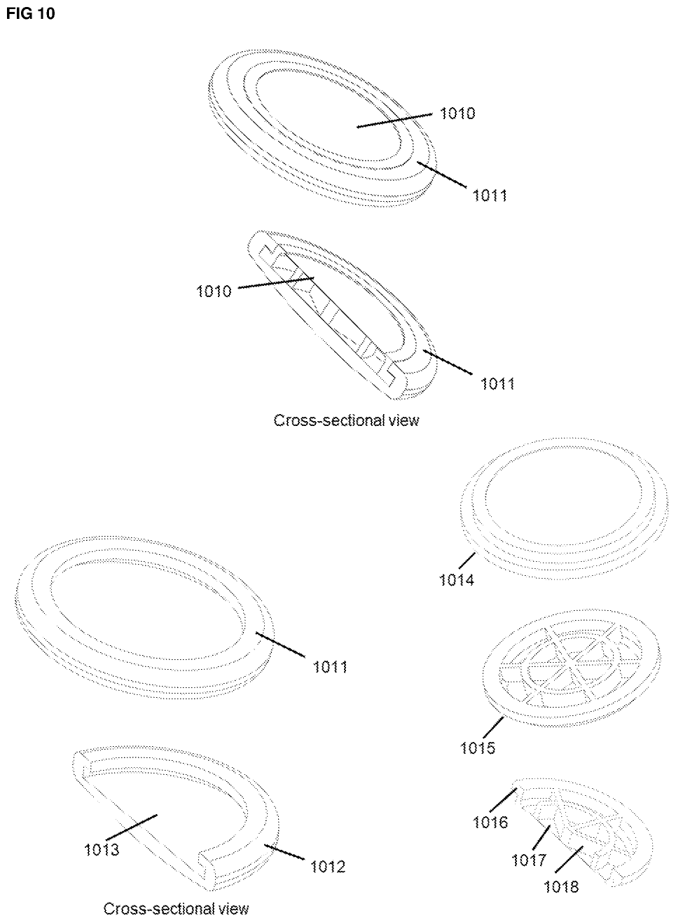

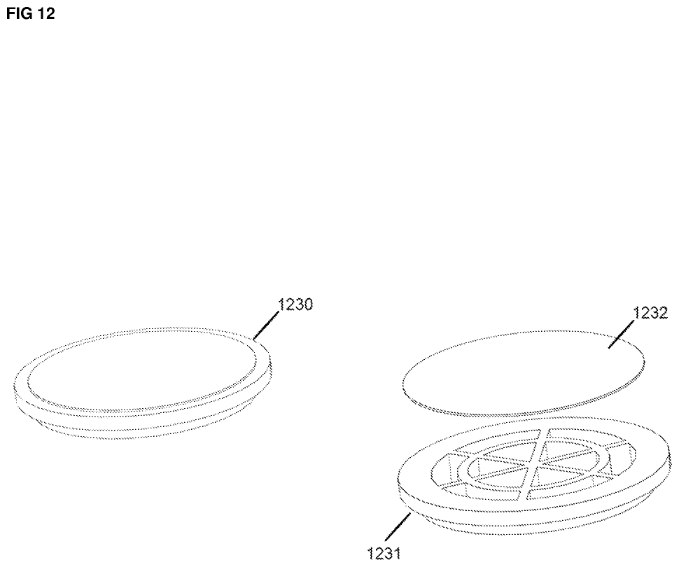

In another embodiment, a capsule-shaped drug delivery device (FIG. 10) comprises a membrane-containing disk, 1010, inserted into an impermeable scaffold, 1011. In some embodiments, the scaffold comprises a sealing ring, 1012, enclosed on one side by an impermeable backing, 1013 to form one or more chambers. In some embodiments, the disk (bottom view, 1014, and top view, 1015) comprises an outer lip, 1016, that fits inside a sealing ring of the scaffold to form a seal; one or more membranes, 1017, that retain cells; and none or one or more rib structures, 1018, that support the membrane and define chambers containing a single membrane region. In some embodiments, the disk may be fabricated as a single part from one material, or it may be assembled, 1230 (FIG. 12), from a first part, 1231, comprising the outer sealing ring and any rib structures and a second part, 1232, comprising a separate membrane that is attached to the first part using adhesive or another assembly method disclosed herein. In some embodiments, cells are retained in one or more chambers formed between the membrane and backing attached to the scaffold, enclosed by the sealing ring.

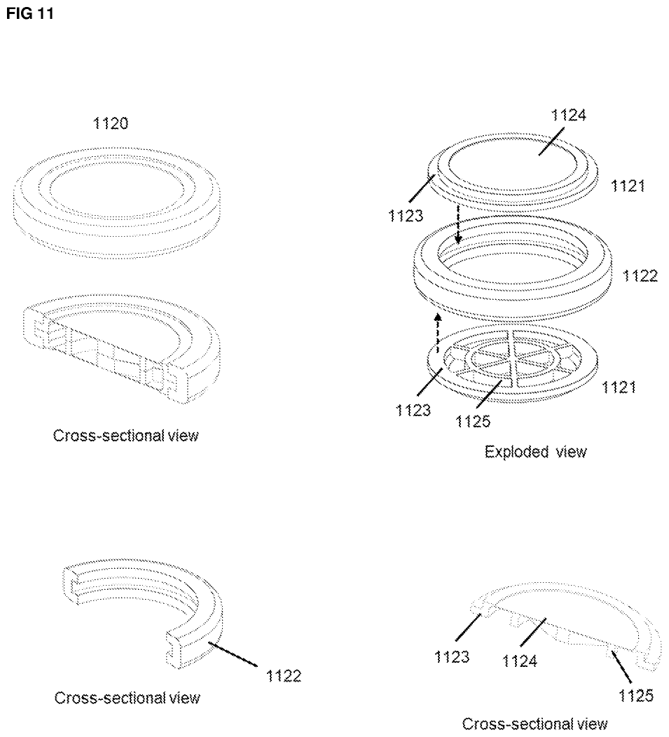

In another embodiment, a capsule-shaped drug delivery device, 1120 (FIG. 11), comprises two membrane-containing disks, 1121, inserted into a drug-impermeable sealing ring, 1122. In some embodiments, the disks comprise an outer lip, 1123, that fits inside the sealing ring to form a seal; one or more membranes, 1124, and none or one or more rib structures, 1125, that support the membrane and define chambers containing a single membrane region. In some embodiments, each disk may be fabricated as a single part from one material, or it may be assembled, 1230 (FIG. 12), from a first part, 1231, comprising the outer sealing ring and any rib structures and a second part, 1232, comprising a separate membrane that is attached to the first part using adhesive or another assembly method disclosed herein. In some embodiments, cells are retained in one or more chambers formed between the two disk structures, and enclosed by the sealing ring, and any rib structures.

### The Drug Delivery Device Chamber (Encapsulation System Reservoir)

The drug delivery device contains a chamber, or a plurality of chambers (see “Encapsulation System Chambers”), that holds living cells. The materials that can make up the chamber body are described above under “Encapsulation Device Materials”. The primary purpose of the drug delivery device chamber is to provide a structure or framework for containing the cells.

### Encapsulation System Chambers

The drug delivery devices disclosed herein comprise one or more chambers comprising a plurality of cells which are held therein. In some embodiments, the device comprises one chamber containing a plurality of cells. In some embodiments, the device comprises more than one chamber containing a plurality of cells.

In some embodiments, the chamber is fully enclosed, e.g., the chamber comprises a continuous wall structure surrounding the cells, such that the cells cannot escape from the chamber. In certain embodiments, the drug delivery device chamber is physically subdivided into multiple chambers. The number of chambers can be one to twelve, or two to twelve, depending on the application. In one embodiment, the chambers are analogous to the camerae of a nautilus shell, separated by septa, or membranes.

In one embodiment, the separation between the chambers is permanent and impermeable. The chambers are isolated from one another and can, for example, contain different cells or cells producing different agents. In these embodiments, multiple agents can be released from one device independently.

In another embodiment, the separation between the chambers is permeable to one or more compounds, e.g., agents, but is made of a non-resorbable material (see “Encapsulation Device Materials”). In some embodiments, the membrane is continuous with no gaps. In other embodiments, there are one or more orifices in the membrane to allow the passage of materials between chambers. In one embodiment, the orifices are between 1 and 1,000 μm in diameter. Non-limiting examples of the embodiments include one or more chambers containing the cells and one or more adjacent chambers that supply nutrients and gases (e.g., oxygen or carbon dioxide) to the cells via the membrane.

In another embodiment, the separation between the chambers is permeable to one or more compounds, but made of a resorbable material (see “Encapsulation Device Materials”). In some embodiments, the membrane is continuous with no gaps. In other embodiments, the membrane comprises one or more orifices to allow the passage of materials between chambers. In one embodiment, the orifices are between 0.01 and 1,000 μm in diameter. Non-limiting examples of the embodiments include one or more chambers containing the cells and one or more adjacent chambers that supply nutrients and gases (e.g., oxygen or carbon dioxide) to the cells via the membrane. In one embodiment, the membrane is impermeable to the flux of nutrients and other compounds and is eroded in vivo over a predetermined period of time. Without wishing to be bound by theory, once the membrane is sufficiently eroded, it allows the passage of materials between the chambers. This approach enables chambers to become connected after the device is used for predetermined periods of time, which has a number of applications.

In one such embodiment, the purpose of a second chamber without cells is to allow expansion of the cells in the first chamber after a predetermined period of time. In another such embodiment, the purpose of a second chamber without cells is to provide one or more nutrients to the cells. In another such embodiment, the purpose of a second chamber without cells is to provide one or more mechanical support structures (e.g., hydrogel) to the cells. In another such embodiment, the purpose of a second chamber without cells is to provide one or more substances that change the metabolism of the cells (e.g., genetic inducer or repressor).

### Microfluidic/Nanofluidic Chambers

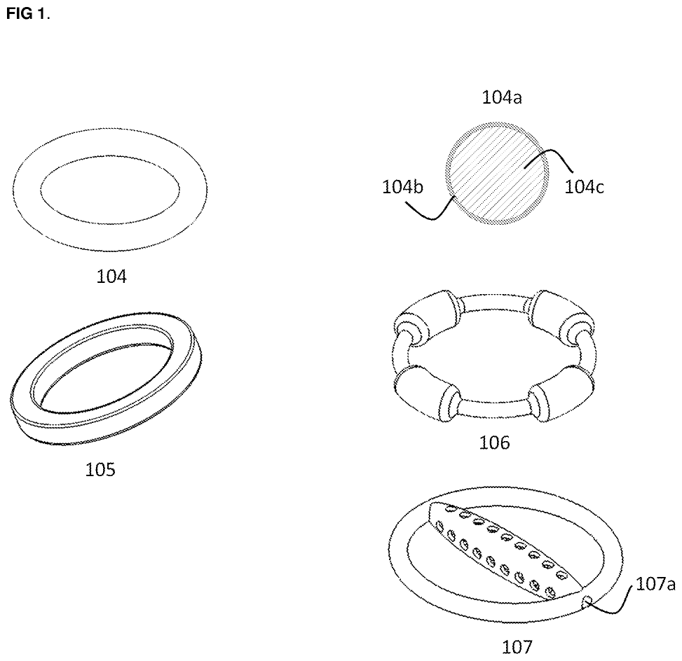

An alternative to using a membrane to retain cells in the drug delivery device and to allow transport of nutrients and oxygen (O₂) to the cells and wastes away from the cells is to use a microfluidic device to isolate cells and control flow into and out of the cell compartment. Microfluidic devices can be in a “chip” format of dimensions 0.1-10 cm 2 (length×width). Chips as described herein can contain micro channels that can be straight lines, or that can form a complex network of features, including chambers or channels, separated by thin walls, membranes or valves. Microfluidic devices have been used in the art for cell culture to control flow of nutrients and wastes to and from cells that are contained in chambers in the microfluidic network. Microfluidic channel dimensions are typically on the order of 10s of μm, a size well-suited for both eukaryotic (10-100 μm) and bacterial (1-10 μm) cells. Microfluidic devices may be used to control the cell microenvironment, including providing nutrients and other soluble factors that regulate cell growth, structure, function, and behavior. A microfluidic “chip” can be designed to take the place of one or two capsules in the drug delivery device structure, for example: at the center of an IVR, 107, (FIG. 1); instead of the two lobes, 312, (FIG. 3); instead of cassette chambers in 700 (FIG. 7) and 800 (FIG. 8); and supported in a pessary structure (FIG. 13).

Microfluidic chips may be fabricated from poly(dimethylsiloxane) (PDMS) or plastic (polymethylmethacrylate, polycarbonate, polystyrene, or cyclic olefin copolymers), alone or in combination, using methods well-established and known to those skilled in the art. In one embodiment, an array of chambers containing cells receives nutrients and O₂ in a liquid flow from a connected chamber. The continuous nutrient flow additionally serves to remove wastes from cellular metabolism along with the active agent(s) produced by the cells, with wastes/agents directed out of the chip and into vaginal fluids through microfluidic channels.

In microfluidic devices, flow is typically controlled using relatively large, external, powered pumps. This is not appropriate for a miniature vaginal device, so flow within the microfluidic device must be controlled passively. One approach is to use pressure to move fluid through microfluidic channels by increasing pressure in a chamber containing fluid (with nutrients, O₂, etc.) and force the fluid into an adjacent chamber connected through microfluidic channels. Pressure can be created by a chemical reaction to produce gas in the chamber. Alternatively, an osmotic pumping mechanism may be used to drive fluid flow. An osmotic gradient forces water from the vaginal cavity to flow across a membrane (e.g., a semipermeable membrane) into an osmotic chamber containing an osmotic agent. The volume of an adjacent chamber separated from the osmotic chamber by a flexible, impermeable elastomer membrane is compressed by the expansion of osmotic chamber volume and provides a pumping force to move liquid from the adjacent chamber through microfluidic channels or a semipermeable separating membrane into the cell compartment. Suitable osmotic agents may include salts, sugars, or polymers such as polyethylene glycol (PEG) and are well-known to one skilled in the art (10).

As an alternative to microfluidic devices that contain multiple cells in a microfluidic compartment, microencapsulation of single cells in synthetic hydrogel microspheres (microgels) may be used to isolate cells but allow transport of nutrients and O₂ into the cell and transport of wastes out of the microgel. The microgel can protect cells from toxins and other harmful substances generated by other encapsulated cells or present in the vaginal compartment, or it can serve to further isolate encapsulated cells and prevent escape from the vaginal encapsulation device. Microgel encapsulated cells can be formed using microfluidic generation techniques (11), incorporated herein in its entirety.

### Supports for the Cellular Cargo Contained in the Vaginal Encapsulation Device

A number of embodiments involving methods and approaches for supporting or dispersing the cellular cargo contained in the drug delivery devices (e.g., vaginal encapsulation devices) are disclosed herein. A support (e.g., a supporting matrix) may be added to the chamber during manufacture of the device (i.e., before the cells are introduced into the chamber), or alternatively the support (e.g., the matrix) may be added to the chamber at the same time as loading of the cells. In some embodiments, the cells may first be combined with the support (matrix, e.g., to porous microbeads), and then the support (matrix) comprising the cells loaded into the chamber.

In some embodiments the cells are enclosed within or disposed on a biocompatible matrix material within the chamber. In some embodiments, the biocompatible matrix material comprises a hydrogel. In some embodiments, the hydrogel is naturally occurring or synthetic. In some embodiments, the hydrogel is resorbable or non-resorbable. Non-limiting examples of suitable matrix materials include polyvinyl alcohol (PVA), alginate, agarose, gelatin, collagen, polyethylene glycol, fibrin, chitosan, and combinations thereof. The matrix may be in the form of, for example, a gel, microbeads, or a sponge. The matrix may be added to the chamber during manufacture of the device (i.e., before the cells are introduced into the chamber), or alternatively the matrix may be added to the chamber at the same time as loading of the cells. In some embodiments, the cells may first be combined with the matrix (e.g., to porous microbeads), and then the matrix comprising the cells loaded into the chamber.

### Hydrogels

In some embodiments the cells may be enclosed within or disposed on a biocompatible matrix material within the chamber, such as a hydrogel. Non-limiting examples of suitable matrix materials include polyvinyl alcohol (PVA), alginate, agarose, gelatin, collagen, polyethylene glycol, fibrin, and chitosan. Three-dimensional hydrogels are used in the art for tissue engineering applications, such as described by Khetan and Burdick (12), incorporated by reference herein in its entirety. In some embodiments, the hydrogel comprises PVA, sodium alginate, hyaluronic acid, PLGA-co-PEG, biomimetic poly(ethylene glycol) gel, or a combination thereof. In some embodiments, the hydrogel comprises PVA-sodium alginate blend, hyaluronic acid-PLGA-co-PEG, or biomimetic poly(ethylene glycol) gel. In some embodiments, the hydrogel comprises PVA. In some embodiments, the hydrogel comprises sodium alginate. In some embodiments, the hydrogel comprises hyaluronic acid. In some embodiments, the hydrogel comprises PLGA-co-PEG. In some embodiments, the hydrogel comprises biomimetic poly(ethylene glycol) gel.

In one embodiment, the cells are encapsulated in a secondary structure that is contained within the drug delivery device. The secondary structure provides a microenvironment that is both protective and stimulatory to cellular health. In one embodiment, the secondary structure comprises one or more biocompatible microspheres. In a non-limiting example, the cells are immobilized in a semipermeable hydrogel that allows bi-directional diffusion of nutrients, O₂, wastes, and secretion of biomolecules.

Hydrogels are well-known in the art for tissue-engineering applications, and these also are useful at maintaining cellular health in the disclosed drug delivery devices. Non-limiting examples have been described in (13-16), incorporated herein by reference in the entirety.

Non-limiting examples of natural polymer-based hydrogels include: Proteins, such as collagen, gelatin, fibrin, silk, lysozyme, Matrigel™, and genetically engineered proteins, such as calmodulin (a calcium-binding protein), elastin-like polypeptides and leucine zipper; Polysaccharides, such as hyaluronic acid (HA), agarose, dextran and chitosan; Protein/polysaccharide hybrid polymers, such as collagen/HA, laminin/cellulose, gelatin/chitosan and fibrin/alginate and DNA.

Non-limiting examples of synthetic polymer-based hydrogels used for this purpose in the art include poly(ethylene glycol) (PEG), 2-hydroxyethyl methacrylate (HEMA), PVA, polyvinylpyrrolidone (PVP), and poly(lactide-co-glycolide) (PLGA)-co-PEG.

Nonbiodegradable synthetic hydrogels can be prepared from the copolymerization of various vinylated monomers or macromers using processes well-known in the art. Non-limiting examples include: 2-hydroxyethyl methacrylate (HEMA), 2-hydroxypropyl methacrylate (HPMA), acrylamide (AAm), acrylic acid (AAc), N-isopropylacrylamide (NIPAm), and methoxyl poly(ethylene glycol) (PEG) monoacrylate (mPEGMA or PEGMA), with crosslinkers, such as N,N′-methylenebis(acrylamide) (MBA), ethylene glycol diacrylate (EGDA) and PEG diacrylate (PEGDA). Another method to form nonbiodegradable hydrogels is to use nonbiodegradable polymers, such as self-assembly of Pluronic® polymers with a structure of poly(ethylene oxide) (PEO)-poly(propylene oxide) (PPO)-PEO, chemical cross-linking of modified PVA, and radiation cross-linking of linear or branched PEG.

Synthetic biodegradable polymers have been extensively studied in the art for tissue-engineering applications. Polyesters are the most widely used biodegradable polymer for scaffold fabrication, including poly(lactic acid) (PLA), poly(glycolic acid) (PGA), poly(ε-caprolactone) (PCL) and their copolymers. They can be used to modify hydrophilic polymers like PEG to form acrylated macromers or amphiphilic polymers for fabricating biodegradable hydrogels via chemical or physical crosslinking. In a non-limiting example, triblock copolymers, PLA-PEG-PLA and PEG-PLA-PEG have been synthesized and end capped with acrylate groups to generate PLA-modified PEG diacrylates. These polyester-containing macromers can be photopolymerized to form hydrolytically degradable hydrogels. In addition, some crosslinkers containing functional groups, such as acetal, ketal, disulfide and poly(propylene fumarate) (PPF), have been used to make biodegradable PEG hydrogels.

The cellular immobilization approach also has the benefit that it makes the cells easy to handle during implant filling.

### Porous Scaffolds

In some embodiments, the interior of the drug delivery device chamber containing the cells comprises a porous support. The support has a porous microstructure (pore sizes 1-1,000 μm). In some embodiments, the support has a porous nanostructure (pore sizes 1-1,000 nm). In yet other embodiments, the support has both porous microstructures and nanostructure. Examples of these microscopic pores include, but are not limited to sponges, including: silica sol-gel materials (17); xerogels (18); mesoporous silicas (19); polymeric microsponges (20); including polydimethylsiloxane (PMDS) sponges (21, 22) and polyurethane foams (23); nanosponges, including cross-linked cyclodextrins (24); and electrospun nanofiber sponges (25) and aerogels (26), all incorporated herein by reference. In some embodiments, the porous sponge comprises silicone, a silica sol-gel material, xerogel, mesoporous silica, polymeric microsponge, polyurethane foam, nanosponge, or aerogel. In some embodiments, the porous sponge comprises silicone. In some embodiments, the porous sponge comprises a silica sol-gel material, xerogel, mesoporous silica, polymeric microsponge, polyurethane foam, nanosponge, or aerogel.

In other embodiments, the capsule interior contains a porous metal structure. Porous metallic materials including, but not limited to, titanium and nickel-titanium (NiTi or Nitinol) alloys in structural forms including foams, tubes, and rods, may be applied as both capsule interior and scaffold materials. Such materials have been used in other applications including bone replacement materials (27-29), filter media (30, 31), and as structural components in aviation and aeronautics (32). These materials have desirable properties for implantation into a body cavity, including resistance to corrosion, low weight, and relatively high mechanical strength. Without wishing to be bound by any particular theory, these properties can be controlled by modifying pore structure and morphology. The pore architecture can be uniform, bimodal, gradient, or honeycomb, and the pores can be open or closed. NiTi alloys additionally have shape-memory properties (ability to recover their original shape from a significant and seemingly plastic deformation when a particular stimulus, such as heat, is applied) and superelastic properties (alloy deforms reversibly by formation of a stress-induced phase under load that becomes unstable and regains its original phase and shape when the load is removed). Without wishing to be bound by any particular theory, for NiTi alloys, these properties are due to transformation between the low-temperature monoclinic allotrope (martensite phase) and high-temperature cubic (austenite) phase. Porous NiTi materials maintain shape memory and/or superelastic properties (33). Both mechanical properties and corrosion resistance are determined by the chemical composition of the titanium alloy. Surface treatment, including chemical treatment, plasma etching, and heat treatment, may be employed to increase or decrease the bioactivity of Ti and Ti-alloy porous materials. Porous Ti metal with 40% in porosity and 300-500 μm pore size was penetrated with newly grown bone more deeply following NaOH and heat treatments (34).

In one embodiment, the cellular compartment comprises a sponge structure, and the cellular suspension is incorporated by impregnation using methods known in the art. In some embodiments, the sponges are magnetic to enable, for example, remotely triggered drug release. See, e.g., (35), incorporated herein by reference.

In one embodiment, the sponge pores are created in situ during use using a templating agent, e.g., a templating particle. A number or porogens are known in the art and have been used to generate porous structures, such as described in (36), incorporated by reference herein in its entirety. As used herein, solid particles can include crystalline or amorphous forms. In one embodiment, the size distribution of the solid particles is polydisperse. In one embodiment, the size distribution of the solid particles is monodisperse. In one embodiment, the solid particles comprise or consist of nanoparticles (mean diameter <100 nm). In one embodiment, the mean diameter of the particles can range from 1-10 nm, 10-25 nm, 25-100 nm, and 100-500 nm. Suitable mean microparticle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, from 400-500 μm, and from 0.5-5 mm. Suitable particle shapes include spheres, needles, rhomboids, cubes, and irregular shapes. In one embodiment, templating particles can comprise salts (e.g., sodium chloride), sugars (e.g., glucose), or other water-soluble excipients known in the art. One skilled in the art would know how to produce such particles of well-defined shape and size.

In one non-limiting embodiment, the porogen particles are fused by exposure to suitable solvent vapors. Particle fusion can be required to result in an open-cell sponge architecture that may be desirable. The fusing solvent can be a polar solvent such as water or an organic solvent with polarities ranging from polar (e.g., methanol) to nonpolar (e.g., hexane), depending on the solubility of the templating agent. The solvent vapors are generated by any suitable method, such as heating, with the column of porogen particles suspended in contact with the vapors using a screen, mesh, or perforated plate, or a suitable container, such as a Buchner funnel with or without a filter. The exposure time can be determined experimentally to achieve the desired degree of particle fusion.

In some embodiments, the pores are formed during manufacture (i.e., prior to use) by immersing the device in a suitable fluid (e.g., water or organic solvent) to dissolve the porogens.

In some embodiments, the pores can form as a result of mechanical, temperature, or pH changes following implantation/use.

In one non-limiting embodiment, the sponge comprises PDMS and the hydrophobic microscopic channels are modified using methods known in the art, such as chemical and plasma treatment. In another embodiment, a linking agent is used between the internal PDMS microchannels and a surface modifying agent to tailor the internal surface properties of the sponge. The surface modifying chemistry is well-known in the art. In one, non-limiting embodiment (3-aminopropyl)triethoxysilane is used as the linking agent and a protein is attached to the PDMS surface as described by Priyadarshani et al. (37), incorporated by reference herein in its entirety.

Provided herein are drug delivery devices wherein the chamber comprises a porous sponge. In some embodiments, the porous sponge comprises silicone, a silica sol-gel material, xerogel, mesoporous silica, polymeric microsponge, polyurethane foam, nanosponge, or aerogel. In some embodiments, the porous sponge comprises silicone. In some embodiments, the porous sponge comprises a silica sol-gel material. In some embodiments, the porous sponge comprises xerogel. In some embodiments, the porous sponge comprises mesoporous silica. In some embodiments, the porous sponge comprises polymeric microsponge. In some embodiments, the porous sponge comprises polyurethane foam. In some embodiments, the porous sponge comprises nanosponge. In some embodiments, the porous sponge comprises aerogel.

In some embodiments, the porous sponge comprises a porogen. In some embodiments, the porogen comprises a fiber mat. In some embodiments, the fiber mat comprises glucose.

### Fiber-Based Supports (Systems)

In another embodiment, a 3D support for the cells may comprise or consist of cellular dispersions in high surface area fiber-based carriers, which are suitable for tissue engineering, delivery of chemotherapeutic agents, and wound management devices, as described in (38), incorporated herein by reference in its entirety. In one embodiment, the high surface area carrier comprises fibers produced by electrospraying. In one embodiment, the high surface area carrier comprises electrospun fibers, including, but not limited to electrospun nanofibers. Electrospun fibers are further described in, for example (39-46), incorporated by reference in their entirety.

Fibers formed by electrospinning may be collected on a plate or other flat surface and chopped, ground, or otherwise reduced in size by methods known in the art to a size that can be effectively packed into the drug delivery device, forming a packed powder that can be premixed with the cells. In an alternative embodiment, the electrospun fibers may be collected on a fixed or stationary collector surface (e.g., a plate or drum) in the form of a mat. The mat may be subsequently cut to an appropriate size and geometry (e.g., cut into strips or sheets), and placed in the device. In yet another embodiment, the fiber support is formed from an electrospun fiber yarn; suitable fabrication methods are described in, e.g., (47-51), incorporated herein by reference in their entirety. In another embodiment, an electrospun fiber support in a cylindrical geometry may be prepared by collecting fibers during the spinning process directly on a rotating wire, fiber, or small diameter mandrel.

Electrospinning may also be used to create a mesh that protects the cells in the drug delivery device from the vaginal environment, while allowing the agent to be released. In one embodiment, a mesh or mat of electrospun fibers is collected on a rotating plate or drum.

The above paragraphs describe embodiments incorporating fibers produced by electrospinning, but additional, non-limiting embodiments use the same approaches incorporating fibers formed by alternative spinning methods. In one embodiment, rotary jet spinning, a perforated reservoir rotating at high speed propels a jet of liquid material outward from the reservoir orifice(s) toward a stationary cylindrical collector surface. The fiber material may be liquefied thermally by melting, resulting in a process analogous to that used in a cotton candy machine, or dissolved in a solvent to allow fiber production at low temperature (i.e., without melting the material). Prior to impaction, the jet stretches, dries, and eventually solidifies to form nanoscale fibers in a mat or bundle on the collector surface.

In another embodiment, fibers may be produced by wet spinning (52) or dry-jet wet-spinning (53, 54) methods. In wet spinning, fibers are formed by extrusion of a polymer solution from a small needle spinneret into a stationary or rotating coagulating bath consisting of a solvent with low polymer solubility, but miscibility with the polymer solution solvent. Dry-jet wet-spinning is a similar process, with initial fiber formation in air prior to collection in the coagulation bath.

Provided herein are devices wherein the support comprises a fiber-based carrier. In some embodiments, the fiber-based carrier comprises an electrospun microfiber or nanofiber. In some embodiments, the fiber-based carrier comprises an electrospun microfiber. In some embodiments, the fiber-based carrier comprises an electrospun nanofiber. In some embodiments, the electrospun nanofiber is a Janus microfiber or nanofiber. In some embodiments, the electrospun nanofiber is a Janus microfiber. In some embodiments, the electrospun nanofiber is a Janus nanofiber.

In some embodiments, the fiber-based carrier comprises random or oriented fibers. In some embodiments, the fiber-based carrier comprises random fibers. In some embodiments, the fiber-based carrier comprises oriented fibers.

In some embodiments, the fiber-based carrier comprises bundles, yarns, woven mats, or non-woven mats of fibers. In some embodiments, the fiber-based carrier comprises bundles, yarns, woven mats, or non-woven mats of fibers. In some embodiments, the fiber-based carrier comprises bundles of fibers. In some embodiments, the fiber-based carrier comprises yarns of fibers. In some embodiments, the fiber-based carrier comprises woven mats of fibers. In some embodiments, the fiber-based carrier comprises non-woven mats of fibers.

In some embodiments, the fiber-based carrier comprises rotary jet spun, wet spun, or dry-jet spun fibers. In some embodiments, the fiber-based carrier comprises rotary jet spun fibers. In some embodiments, the fiber-based carrier comprises wet spun fibers. In some embodiments, the fiber-based carrier comprises dry-jet spun fibers.

In some embodiments, the polymer material used to build the fiber-based scaffold comprises a resorbable or non-resorbable polymer material described herein, e.g., poly(dimethyl siloxane), silicone, a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof. In some embodiments, the polymer comprises expanded poly(tetrafluoroethylene) (ePTFE) or ethylene vinyl acetate (EVA). In some embodiments, the polymer comprises expanded poly(tetrafluoroethylene) (ePTFE). In some embodiments, the polymer is ethylene vinyl acetate (EVA). In some embodiments, the polymer comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof.

In one embodiment, the polymeric fibers are medical-grade and commercially available. For example, Zeus Bioweb™ (Zeus Industrial Products, Inc., Orangeburg, SC) consists of PTFE electrospun into polymeric fibers with diameters ranging from nano- to microscale. Collectively, these electrospun filaments form materials with a broad range of fiber and fabric properties.

### The Cellular Cargo of the Drug Delivery Device (Vaginal Encapsulation Device)

The drug delivery devices (e.g., vaginal encapsulation devices) described herein can be used to encapsulate any type of living cell for the in vivo production and release of one or more pharmaceutically active substances, e.g., actives, to the vaginal mucosa. Non-limiting examples of suitable cells include bacterial (e.g., Lactobacillus spp.), archaeal, fungal (e.g., yeast strains), plant, and animal, including mammalian cells, and including human cells. The cells can be “wild type” (i.e., genetically unmodified) or genetically modified (i.e., recombinant).

The drug delivery systems disclosed herein comprise one or more chambers containing a plurality of cells In some embodiments, the chamber or chambers comprise a plurality of cells of the same type. In some embodiments, the chamber or chambers comprise a plurality of cells of different types. In some embodiments, the chamber or chambers comprise a plurality of cells of two or more types. In some embodiments, the chamber or chambers comprise a plurality of cells of two types. In some embodiments, the chamber or chambers comprise a plurality of cells of three types.

In one embodiment, the encapsulated cells are selected based on their natural occurrence vaginally, so that they already are adapted to the vaginal milieu. Non-limiting examples include: bacteria, such as Lactobacillus spp., Gardnerella spp. and Pseudomonas spp.; fungi, such as Candida spp.; and human cells, such as vaginal and cervical epithelial cells.

In some embodiments, the plurality of cells comprises bacterial cells, fungal cells, mammalian cells, or a combination thereof. In some embodiments, the plurality of cells comprises bacterial cells. In some embodiments, the bacterial cells comprise one type of bacterial cell. In some embodiments, the bacterial cells comprise a combination of bacterial cells. In some embodiments, the bacterial cells comprise one or more members of the Lactobacillus genus. In some embodiments, the bacterial cells comprise Lactobacillus crispatus cells, L. gasseri cells, L. jensenii cells, L. rhamnosus cells, L. iners cells, or a combination thereof. In some embodiments, the bacterial cells comprise Lactobacillus crispatus cells. In some embodiments, the bacterial cells comprise L. gasseri cells. In some embodiments, the bacterial cells comprise L. jensenii cells. In some embodiments, the bacterial cells comprise L. rhamnosus cells. In some embodiments, the bacterial cells comprise L. iners cells. In some embodiments, the plurality of cells comprises fungal cells. In some embodiments, the fungal cells comprise yeast cells. In some embodiments, the plurality of cells comprises Candida albicans cells. In some embodiments, the plurality of cells comprises Candida albicans cells of vaginal origin. In some embodiments, the plurality of cells comprises mammalian cells. In some embodiments, the mammalian cells comprise non-human mammalian cells. In some embodiments, the mammalian cells comprise mouse microglial cells, mouse myoblast cells, Chinese hamster ovary cells, or a combination thereof. In some embodiments, the mammalian cells comprise human cells. In some embodiments, the human cells comprise human embryonic kidney cells, cervicovaginal epithelial cells, THP-1 monocyte cells, or a combination thereof. In some embodiments, the human cells comprise human embryonic kidney cells. In some embodiments, the human cells comprise cervicovaginal epithelial cells. In some embodiments, the human cells comprise THP-1 monocyte cells. In some embodiments, the plurality of cells comprises wild type cells. In some embodiments, the plurality of cells comprises recombinant cells. In some embodiments, the plurality of cells comprises C8-B4 mouse microglial cells, recombinant C2C12 cells, recombinant Chinese hamster ovary cells, recombinant human embryonic kidney 293 (HEK-293) cells, 293LTV cells, recombinant cervicovaginal epithelial cells, recombinant THP-1 monocyte cells, or a combination thereof.

In some embodiments, the plurality of cells produce one or more agents. As used herein, the term “agent” refers to a pharmaceutically active substance produced by cells disclosed herein. Non-limiting examples of pharmaceutically active substances produced by cells disclosed herein include: peptides and proteins, including antibodies and lectins; nucleic acid polymers (e.g., oligomers), including messenger ribonucleic acids (mRNAs)—synthetic or natural—to stimulate the in vivo expression of one or more proteins (55), such as antibodies (56) and vaccine adjuvants (57) in the vaginal mucosa (i.e., by vaginal epithelial cells or other cell types present in the vaginal mucosa); other biopolymers (e.g., polysaccharides); small molecule agents, ranging from prebiotic compounds, to pharmaceutical agents, to naturally produced agents such as cannabinoids. In some embodiments, the cells produce single-stranded or double-stranded nucleic acids. In some embodiments, the one or more agents comprise vaccines, peptides, proteins, nucleic acids, or small molecules. In some embodiments, the one or more agents comprise one or more small molecules. In some embodiments, the one or more small molecules comprise lactic acid. In some embodiments, the one or more agents comprise one or more proteins. In some embodiments, the one or more proteins comprise an antiviral protein. In some embodiments, the antiviral protein comprises a lectin. In some embodiments, the one or more agents comprise one or more peptides. In some embodiments, the one or more peptides comprises an antiviral peptide, an anti-cancer peptide, a messenger RNA (mRNA), or combinations thereof.

In some embodiments, the one or more proteins comprise one or more antibodies, antibody fragments, or nanobodies. In some embodiments, In some embodiments, the one or more proteins comprise one or more antibodies. The term “antibody” refers to an intact antigen-binding immunoglobulin. The antibody can be an IgA, IgD, IgE, IgG, or IgM antibody, including any one of IgG1, IgG2, IgG3 or IgG4. In various embodiments, an intact antibody comprises two full-length heavy chains and two full-length light chains. In some embodiments, the one or more proteins comprise one or more antibody fragments, preferably antigen-binding antibody fragments. In some embodiments, the one or more proteins comprise an Fc region of an IgG1 or IgG3 antibody. In some embodiments, the one or more proteins comprise one or more nanobodies. In some embodiments, the one or more antibodies comprise a nonhormonal contraceptive comprising immunoglobulin G (IgGs) which trap sperm. In some embodiments, the one or more agents comprise a neutralizing antibody. In some embodiments, the neutralizing antibody is an HIV-neutralizing antibody.

The architecture of antibodies has been exploited to create a growing range of alternative formats that that mimic antibody fragments which retain full antigen-binding capacity, e.g., scFvs, Fabs and VHH. Examples of these types of antibody-like products include, but are not limited to, scFv (single chain fragment variable), disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. Other antibody-like products include a single chain antibody (SCA); a diabody; a triabody; a tetrabody; bispecific or trispecific antibodies, and the like. These alternative format, antibody-like products, also are contemplated for use in the context of the disclosure.

In some embodiments, the wild type or genetically modified cells express one or more pharmaceutically active peptides. Non-limiting examples of such peptides include: antiviral fusion inhibitors such as enfuvirtide, and derivatives thereof; broad antiviral peptides such as described by Cheng et al. (58), incorporated herein its entirety; peptides known in the art to treat or manage diabetes mellitus including, but not limited to exenatide, octreotide, goserelin, or derivatives and pharmaceutically acceptable salts thereof; and the GnRH agonist leuprolide, or derivatives and pharmaceutically acceptable salts thereof, used in the management and treatment of prostate cancer, endometriosis, uterine fibroids, precocious puberty, and other sex hormone-related conditions.

In some embodiments, the wild type or genetically modified cells express one or more pharmaceutically active proteins. In one non-limiting example, the antiviral protein belongs to the lectin family, such as griffithsin (GRFT), cyanovirin-N (CV-N) and scytovirin (SVN) (59), incorporated herein in its entirety. In another non-limiting example, the antiviral agent produced by the recombinant cells consists of a broadly neutralizing antibody (bNAb), such as VRC01 that possesses activity against various HIV-1 isolates (60). Other, next generation bNAbs that are more potent against HIV-1 or neutralize other viruses such as HSV or HPV, or bacteria, such as multidrug-resistant Neisseria gonorrhoeae, such as described in the art, incorporated herein by reference in its entirety (61-64). In another embodiment, the cells produce a nonhormonal contraceptive consisting of multivalent IgGs with high agglutination potencies for trapping vigorously motile sperm (65, 66).

In another embodiment, delivery of one or more agents secreted by the cells contained in the vaginal device leads to immunization of the host. Most infections affect or start from a mucosal surface, and that in these infections, topical application(s) of a vaccine is often required to induce a protective immune response. Examples include gastrointestinal infections caused by Helicobacter pylori, Vibrio cholerae, enterotoxigenic Escherichia coli (ETEC), Shigella spp., Clostridium difficile, rotaviruses and caliciviruses; respiratory infections caused by Mycoplasma pneumoniae, influenza virus and respiratory syncytial virus; and sexually transmitted genital infections caused by HIV, HSV, Chlamydia trachomatis, and Neisseria gonorrhoeae (vide infra). Attenuated, or modified antigen and adjuvants secreted from an encapsulated device and absorbed through mucosae can initiate/enhance immunogenic responses. In some embodiments, a mucosal vaccine is delivered from the encapsulated cells. Production of these recombinant molecules is controlled through an on-demand expression system to eliminate tolerance. Mucosal vaccines are known in the art; e.g., against HSV as described by Oh and Iwasaki (67), incorporated herein by reference in its entirety. In a non-limiting example, a recombinant protein product of a modified bacterial toxin (e.g., cytolysin) with inactivated active sites and conserved epitopes to induce a controlled immune response is secreted from an expression system (e.g., pERV3, pEGSH) in the encapsulated cells. In another example of the embodiment, a large protein or a subunit (e.g., SerpinA1) is expressed by the cells.

In various aspects, the cells disclosed herein produce bacteriophages (or phages), bacterial viruses capable of invading bacterial cells and, optionally, killing the infected bacterial cells. Phages are suitable for delivering payloads to bacterial cells as well as causing bacterial cell lysis. In the context of lytic phages, phage therapy may be used as an alternative to traditional antibiotics, as phages target particular bacteria, providing a more personalized approach to treating infection. Further, phage therapy can be effective in treating multi-drug resistant bacterial infections. Phages may also be employed to adjust the microbiome of target tissues. In this aspect of the disclosure, the cells support the replication and/or delivery of phage to the target anatomical site.

### Cellular Inducers/Restrictors

Mammalian cells are capable of protein folding and post-translational modification, which can express proteins with molecular structures, physical and chemical structures, and biological functions. These molecules may include antibody-based drugs, anticoagulants, blood factors, bone morphogenetic proteins, engineered protein scaffolds, enzymes, Fc fusion proteins, growth factors, hormones, interferons, interleukins, and thrombolytics. In one embodiment, well-defined cell lines (e.g., CHO, HEK) are transfected or transduced with a protein expression system (e.g., pERV3, pEGSH) carrying a gene construct to produce stable cell lines that can express gene products either on demand or constitutively. The expression method is determined based on the promotor used with such gene constructs. An inducible, on-demand promoter allows to control the timing of gene expression. Introduction of a molecule (steroid analog; e.g., Ecdysone or synthetic molecule; e.g., IPTG) that influence the promotor/receptor region will either induce or repress the transcription of the gene segment respectively, leading to increased or decreased expression of the gene product of interest in this application. The artificial nature of the receptor and the recognition element ensures that endogenous host transcription factors and genes are not activated.

The Drug Delivery Device (Encapsulation System) Membrane

The drug delivery devices disclosed herein comprise one or more membranes that prevent cells from escaping the device, but permit the release of one or more agents to the vaginal fluids, mucosa, and tissues. The drug delivery devices disclosed herein comprise one or more membranes, made up of one or more biocompatible materials described above under “Encapsulation Device Materials” which are permeable to agents produced by the cells contained in the drug delivery devices. The primary purpose of the membrane is to contain the living cells in the device and prevent their release, while being at least partially permeable to agents produced by the cells. In certain embodiments, the membrane(s) allow the diffusion of cellular nutrients, including gases such as oxygen and carbon dioxide, into the device while also allowing cellular waste materials out of the device.

In some embodiments, the drug delivery device comprises one membrane. In some embodiments, the drug delivery device comprises more than one membrane.

In some embodiments, one or more chambers of the devices disclosed herein are disposed between one or more membranes comprising semipermeable layers. Each semipermeable layer may be comprised of one or more sub-layers, e.g., each semipermeable layer may comprise a laminar structure. In some embodiments, only one of the sub-layers is semipermeable. In some embodiments, the membrane or membranes are semipermeable.

In one embodiment, the membranes have a pore size such that oxygen and other molecules important to cell survival and function can move through the membranes, but the cells (e.g., the encapsulated cells and/or cells of the host immune system) cannot permeate or traverse through the pores.

In embodiments where the device further encapsulates one or more agents or cells that produce one or more agents (e.g., an antibody, a growth factor, or a hormone) the membrane can allow the agent of interest to pass through the layer, from the chamber of the device into the surrounding tissue, in order to provide access to the target cells outside the device in the host vaginal tissue or organism.

In some embodiments, the membrane (e.g., a membrane comprising a semipermeable layer) allows one or more nutrients present in the subject to pass through the layer to provide essential nutrients to the encapsulated cells. For example, in one embodiment, the semipermeable layer allows glucose and oxygen to stimulate the encapsulated cells to release the target agent, while preventing immune system cells from recognizing and destroying the implanted cells. In some embodiments, the membrane prohibits the implanted cells from escaping encapsulation.

In some embodiments, the membrane or membranes comprise a biocompatible material. In some embodiments, the membrane (e.g., a semipermeable layer or one or more sub-layers thereof) comprises a biocompatible material that functions under vaginal physiologic conditions, particularly physiological pH and temperature. Non-limiting examples of materials that may be used in the membrane include polyester, polypropylene, polycarbonate, polyethylene terephthalate (PET), anisotropic materials, polysulfone (PSF), microfiber and nanofiber mats, polyimide, tetrafluoroethylene/polytetrafluoroethylene (PTFE; also known as Teflon®), expanded polytetrafluoroethylene (ePTFE), polyacrylonitrile, polyethersulfone, acrylic resin, cellulose acetate, cellulose nitrate, polyamide, hydroxylpropyl methyl cellulose (HPMC), and combinations thereof. Suitable materials known to those skilled in the art are manufactured by, for example, Gore®, Phillips Scientific®, Zeus®, Pall® and Dewal®. In some embodiments, the membrane or membranes comprise polyester, polypropylene, polycarbonate, polyethylene terephthalate (PET), anisotropic materials, polysulfone (PSF), microfiber or nanofiber mats, polyimide, tetrafluoroethylene/polytetrafluoroethylene (PTFE), expanded polytetrafluoroethylene (ePTFE), poly(ethylene-co-vinyl acetate) (EVA), polyacrylonitrile, polyethersulfone, acrylic resin, cellulose acetate, cellulose nitrate, polyamide, hydroxylpropyl methyl cellulose (HPMC), or a combination thereof. In some embodiments, the membrane or membranes comprise ePTFE, PTFE, polypropylene, poly(ethylene-co-vinyl acetate) (EVA), cellulose acetate, or a combination thereof. In some embodiments, the membrane or membranes comprise polyester. In some embodiments, the membrane or membranes comprise polypropylene. In some embodiments, the membrane or membranes comprise polycarbonate. In some embodiments, the membrane or membranes comprise polyethylene terephthalate (PET). In some embodiments, the membrane or membranes comprise anisotropic materials. In some embodiments, the membrane or membranes comprise polysulfone (PSF). In some embodiments, the membrane or membranes comprise microfiber or nanofiber mats. In some embodiments, the membrane or membranes comprise polyimide. In some embodiments, the membrane or membranes comprise tetrafluoroethylene/polytetrafluoroethylene (PTFE). In some embodiments, the membrane or membranes comprise expanded polytetrafluoroethylene (ePTFE). In some embodiments, the membrane or membranes comprise poly(ethylene-co-vinyl acetate) (EVA). In some embodiments, the membrane or membranes comprise polyacrylonitrile. In some embodiments, the membrane or membranes comprise polyethersulfone. In some embodiments, the membrane or membranes comprise acrylic resin. In some embodiments, the membrane or membranes comprise cellulose acetate. In some embodiments, the membrane or membranes comprise cellulose nitrate. In some embodiments, the membrane or membranes comprise polyamide. In some embodiments, the membrane or membranes comprise hydroxylpropyl methyl cellulose (HPMC).

In some embodiments, the semipermeable layer is chemically inert or non-toxic with respect to the cells encapsulated inside the device and/or the host tissue or organism. The membrane (e.g., a membrane comprising a semipermeable layer) can permit secretion or release of a biologically active agent (produced by the encapsulated cells or encapsulated with the cells) across the device, and can promote rapid kinetics of macromolecule diffusion with no or minimal slowing of release to minimize the buildup of the agent in the device. In further embodiments, the semipermeable layer may promote long-term stability of the encapsulated cells.

In some embodiments, the membrane (e.g., a membrane comprising a semipermeable layer) comprises two or more sub-layers; e.g., is a laminar structure. For instance, the membrane (e.g., a membrane comprising a semipermeable layer) may be a laminated structure comprising 2, 3 or 4 sub-layers. In one embodiment, the membrane (e.g., a membrane comprising a semipermeable layer) comprises a first sub-layer that is in contact with the chamber and a second sub-layer that overlies the first sub-layer to the exterior of chamber; i.e., the first sub-layer lies on the inside and the second sub-layer lies on the outside of the device.

In some embodiments, the membrane is compatible with the cells inside the chamber, and that has a pore size which permits solute transport but prevents entry or egress of cells. In some embodiments, the pore size of the membrane is less than about 2 μm (i.e., 2 micrometers), in order to prevent the ingress of vascular structures. Pore sizes less than about 0.6 μm are preferred is some embodiments, in order to prevent the access of cells—including macrophages, foreign body giant cells, and fibroblasts—to the internal chamber of the device. “Pore size” designates the maximum pore size of the material. Pore size may be determined using conventional bubble point methodology, or other methods known in the art.

In one embodiment, the membrane comprises polypropylene, polycarbonate, PET, or PTFE. For instance, in one embodiment, a semipermeable PTFE membrane material having a thickness of about 25 μm and a maximum pore size of about 0.4 μm is used. Alternative materials for the membrane layer include polyethylene, cellulose acetate, cellulose nitrate, polyester, nylon, polysulfone materials, cellulose, polyvinylidene difluoride, acrylic, silicone, and polyacrylonitrile.

In one embodiment, the membrane comprises a mesh layer, which typically serves to provide mechanical support or mechanical protection to the first sublayer. Accordingly, the membrane preferably comprises a mesh having an average nominal pore size of approximately 0.01 to about 1 mm (i.e., 0.01 to 1 millimeters). Preferably, at least approximately 50% of the pores of the membrane have an average size of approximately 0.01 to about 1 mm.

In some embodiments, the mesh layer is formed from fibers or strands or a polymer material. These strands are typically elongated structures having one dimension much larger than the other two. Suitable materials for the mesh layer include nylon, polyester, and PTFE. In one embodiment the mesh layer is made from nylon and has a pore size of approximately 0.12 mm. Further suitable materials are disclosed, for example, in (68), incorporated herein in its entirety.

In one embodiment, a membrane—e.g., a semipermeable membrane that excludes the ingress/egress of cells, but allows for the diffusion of agents, including proteins—is composed of patterned microscopic channels, different from the porous materials described above. In some embodiments, the membranes are produced by methods known in the art. Non-limiting examples of porous, patterned membranes include: nanofluidic membrane produced using semiconductor fabrication techniques known in the art (69); nanostructured polymer membrane using a template-based approach, such as zinc oxide nanorods (70); and nanostructured/microstructured membranes fabricated by photolithography (71). These non-limiting examples are incorporated herein in their entirety

Laminated semipermeable structures for use in cell encapsulation have been described; e.g., in (72, 73). For instance, an ePTFE membrane material may be laminated with a BIOPORE™ membrane material—that serves as an immune-isolation membrane for allografts—using a crisscrossing pattern of non-permeable polymeric adhesive.

### Nutrient Supplementation Systems (Means of Providing Cellular Nutrients)

Cellular implants have been placed surgically in the subcutaneous environment. The subcutaneous anatomic location has the benefit that it can readily lead to dense neovascularization of the devices, as shown by Lathuiliere et al. (74), incorporated herein in its entirety by reference. Vascularization holds the benefit that it can aid in the transport of nutrients, including dissolved gases such as oxygen, to the implanted cells, while removing waste products. When a cellular implant is placed in the vagina, a mucosal tissue surface, the beneficial potential for neovascularization largely is lost.

The drug delivery devices described herein comprise one or more nutrient supplementation systems. As used herein, “nutrient supplementation system” refers to an element of the drug delivery device intended to provide one or more nutrients required to sustain and/or enable growth of the cells of the device. In some embodiments, the nutrient supplementation system comprises nutrients and/or growth factors required for growth and/or maintenance of cells of a drug delivery device disclosed herein. In one embodiment, nutrients and growth factors are added with the cells to the drug delivery device during fabrication. Contemplated agents which maintain cellular health include, but are not limited to: Bacteria: peptone or casein-digested products (e.g., pancreatic digest of casein), monosaccharides (e.g., glucose, fructose), yeast extract, inorganic salts (e.g., magnesium sulfate, calcium chloride, potassium dihydrogen phosphate), and surfactants (e.g., polysorbate 80); Fungi: peptone, yeast extract, monosaccharides (e.g., dextrose, glucose), amino acids, vitamins [e.g., thiamine (B₁), riboflavin (B₂) niacin (B₄), pyridoxine (B₆), biotin (B₇)], calcium pantothenate, coenzyme A, thiamine pyrophosphate, and inositol; Mammalian cells: amino acids, vitamins, inorganic salts (for pH control, osmotic balance, and regulation of membrane potential), carbohydrates (e.g., glucose), lipids (e.g., cholesterol, steroids, fatty acids, prostaglandins), polyamines, reductants (e.g., 2-mercaptoethanol), protective agents (e.g., carboxymethyl cellulose, polyvinyl pyrrolidone), surfactants (e.g., polysorbate 80), hormones/growth factors (e.g., somatostatin, hydrocortisone, insulin thyrotropin, platelet-derived growth factor, fibroblast growth factor, progesterone, estradiol, neurotensin, luteinizing hormone, aldosterone, thyroxine), procoagulants/anticoagulants (e.g., thrombin), and transferrin. In some embodiments, the one or more nutrient supplementation systems comprise nutrients, growth factors, hormones, vitamins, O₂-generating agents, pH buffering agents, cell culture media, antibiotics, or a combination thereof. In some embodiments, the one or more nutrient supplementation systems comprise nutrients. In some embodiments, the one or more nutrient supplementation systems comprise growth factors. In some embodiments, the one or more nutrient supplementation systems comprise hormones. In some embodiments, the one or more nutrient supplementation systems comprise vitamins. In some embodiments, the one or more nutrient supplementation systems comprise O₂-generating agents. In some embodiments, the one or more nutrient supplementation systems comprise pH buffering agents. In some embodiments, the one or more nutrient supplementation systems comprise cell culture media. In some embodiments, the one or more nutrient supplementation systems comprise antibiotics. In some embodiments, the one or more nutrient supplementation systems comprise glucose, glycogen, peptone, amino acids, proteins, antibiotics, or a combination thereof. In some embodiments, the one or more nutrient supplementation systems comprise glucose. In some embodiments, the one or more nutrient supplementation systems comprise glycogen. In some embodiments, the one or more nutrient supplementation systems comprise peptone. In some embodiments, the one or more nutrient supplementation systems comprise amino acids. In some embodiments, the one or more nutrient supplementation systems comprise proteins. In some embodiments, the one or more nutrient supplementation systems comprise D-glucose-glycogen, 2:1 w/w; or D-glucose-peptone, 2:1 w/w. In some embodiments, the one or more nutrient supplementation systems comprise RPMI 1640, L-glutamine, HEPES, heat-inactivated fetal bovine serum, Normocin™, and Pen-Strep.

These agents are readily depleted from drug delivery devices in vivo. To increase the duration of nutrient supply from the implant interior, several strategies have been developed. In one embodiment, the nutrient(s) is formulated as a time-release additive. Non-limiting examples include incorporation into resorbable and non-resorbable beads or particles where the nutrient is slowly released over time. Methods for producing time-release formulations of compounds are well known in the art and include: polymer coating of cores containing the compound(s); incorporation of the compound(s) into polymer matrices by extrusion or coacervation; and spray-drying with suitable excipients. In some embodiments, the one or more nutrient supplementation systems comprise a time-release additive. In some embodiments, the time-release additive comprises PLGA-coated beads.

In one embodiment, the agent(s) supporting cellular growth and health are released in vivo from micro- or nanofibers, as described above for scaffolds. In one example, the agent is delivered from electrospun fibers. Electrospun, fibers containing nutrients and other compounds useful for cellular growth and health can have a number of configurations. For example, in one embodiment, the agent is embedded in the fiber (75). In another embodiment, the agent-fiber system is produced by coaxial electrospinning to give a core-shell structure (76, 77). Core-shell fibers production by coaxial electrospinning produces encapsulation of water-soluble agents, such as biomolecules including, but not limited to proteins, peptides, and the like (78). In yet another embodiment, Janus nanofibers can be prepared; exemplary suitable methods are described in (79). Janus fibers contain two or more separate surfaces having distinct physical or chemical properties, the simplest case being two fibers joined along an edge coaxially. In some embodiments, it may be advantageous to modify the fibers by surface-functionalization, as described in, e.g., (80, 81), incorporated herein by reference in its entirety.

In one embodiment, the agent (i.e., nutrient or agent beneficial to cellular growth and health) is shaped into nanofibers directly, there-by fulfilling the dual purpose of scaffold and nourishing the cells. In one embodiment, a process, described above, consists of rotary jet spinning. In some embodiments, the fiber material consists of a pharmaceutically acceptable excipient, such as glucose or sucrose, or a polymer material e.g., a resorbable or non-resorbable polymer described herein. In another embodiment, the solid excipient(s) or polymer are premixed as solids and formed into a fiber mat by spinning. Rotary jet spinning methods are known in the art, for example (82-85), incorporated by reference in their entirety.

In some embodiments, the fiber comprises glucose, sucrose, or a polymer material. In some embodiments, the fiber comprises glucose. In some embodiments, the fiber comprises sucrose. In some embodiments, the fiber comprises a polymer material.

In another embodiment, agents used to promote cellular growth and health, either unformulated or formulated as described above, are loaded into one or more device chambers that are separated from a main device chamber that is initially in contact with the vaginal environment.

In another embodiment, agents used to regulate or buffer pH known in the art including inorganic salts (e.g., monobasic/dibasic phosphate salts, carbonate/bicarbonate salts, borate salts, Hanks' balanced salts, Dulbecco's phosphate buffered saline) and organic systems (e.g., acetate salts, HEPES, Tris-EDTA, MOPS, MES) either unformulated or formulated as described above, are loaded into one or more device chambers that are separated from a main chamber that is initially in contact with the vaginal environment.

### Means of Generating Gaseous Compounds

In certain applications, it may be necessary to provide for the in situ generation of gases as part of the drug delivery device. The gas-generating substances are either liquid or solid and can be mixed with the cellular formulation at device filling. In another embodiment, the gas-generating substances and, hence, the release of gas, are delivered over time from systems analogous to those described above for nutrients and cellular health-supporting compounds.

In one embodiment, the gas-generating substance produces carbon dioxide, CO₂. In one nonlimiting example, the CO₂-generating substance comprises a suitable carbonate or bicarbonate salt. In another embodiment, the CO₂-generating substance is co-formulated, but separated, with an acid that helps liberate the CO₂ when the two substances come in physical contact. In some embodiments, the acid comprises a short-chain fatty acid. In one example, the acid is comprised of DL-lactic acid, D-lactic acid, or L-lactic acid. The soluble species H₂CO₃, HCO₃⁻, and CO₃²⁻ are in equilibrium, from the decomposition of carbonate salts in the presence of an acidic environment.

In another embodiment, the gas-generating substance produces O₂. The vagina is microaerophilic; a high CO₂, low O₂ environment that complicates the growth of certain cell lines. For cell lines that cannot thrive in an anoxic environment, a component for supplying or generating O₂ may be included in the device. One approach is to include an O₂ storage or generation system in the main cell compartment, or in a separate chamber adjacent to a chamber containing cells and separated by an oxygen permeable membrane.

An O₂ carrier approach is to conjugate or encapsulate hemoglobin (HB) into hybrid polymeric oxygen carriers to store and transport O₂ to the cells. Hemoglobin can be conjugated to a polymer carrier such as gluteraldehyde-polymerized HB (86) or poly(ethylene glycol)-conjugated HB (PEG-HB) (87), included by reference in their entirety. The polymer carrier can act to trap the HB in the cell chamber or an adjacent chamber separated from the cell chamber by an O₂ permeable membrane or other suitable barrier to allow diffusion of O₂ to the cells but prevent escape of the HB conjugate. An alternative to polymer conjugation is encapsulation of HB in lysosomes or lipid-polymer membrane structures to retain the HB in the device, enhance the HB-02 lifetime and reduce side effects similar to the approach used in treatment of hypoxic tumors (88-90), included by reference in their entirety.

Non-HB carriers can also be used instead of HB. Non-HB oxygen carriers include perfluorohexane (91), perfluorotributylamine (92), perfluorooctyl bromide (93), perfluoropropane (94), or perfluoropentane (95). The perfluorocarbon O₂ carrier may be encapsulated in a core-shell nanostructure with the carrier in the core and a lipid or other protective shell surrounding the perfluorocarbon.

An alternative approach is to use oxygen-loaded microbubbles or nanobubbles to provide O₂ in the hypoxic vaginal environment. Similar methods have been used to supply O₂ to hypoxic tumors (96), incorporated herein. Oxygen nanobubbles can consist of an oxygen core enclosed in a layered shell including lipid, polymer, dextran, and gas vesicles, as known in the art (97, 98), incorporated herein in their entirety. Nanobubbles can be freeze dried for storage and incorporation into a cell encapsulation device.

Catalytic decomposition of hydrogen peroxide (H₂O₂) naturally occurring in the vagina or released from a component of the device may be used to form O₂ in situ. Catalase (CAT) and CAT-like nanozymes may be used to decompose H₂O₂ to O₂ in the encapsulation device. Natural CAT is an efficient catalyst for H₂O₂, but it exhibits a short half-life and is often unstable under in vivo conditions due to proteolytic degradation. Stabilization of CAT by loading into a metal organic framework (MOF) has been used to preserve CAT from degradation in vivo, for example (99, 100), incorporated herein in their entirety. CAT can also be incorporated into polymers such as, but not limited to, hyaluronic acid (101), fluorinated polyethyleneimine (102), poly(ethylene glycol) double acrylate (103), and modified chitosan (104), all incorporated herein in their entirety. CAT can also be stabilized by incorporation into inorganic nanoparticles formed from oxides of tantalum, titanium, silicon, manganese, aluminum, or iron oxides, singly or in combination, as described in (105), incorporated herein in its entirety.

Nanoparticles can be combined with polymers to form nanomaterials that exhibit enzyme-like properties such as H₂O₂ decomposition and are known in the art as “nanozymes” (106). For example, H—MnO₂-PEG nanoparticles can decompose H₂O₂ to O₂ (107). A large number of nanomaterials show catalase-like activity, including diamond; fullerene (C60); graphene oxide quantum dots; N-doped mesoporous carbon; nanoparticles and nanorods of Au, Au/Cu, Au/Pt, Ag/Pt,A Ag/Au/Pt; metal oxides (CeO₂, CoFe₂O₄, CuFe₂O₄, CuO, Fe₂O₃, Fe₃O₄, IrO₂, LaCoO₃, MgFe₂O₄, MnFe₂O₄, MnO₂, Mn₃O₄, NiFe₂O₄, RuO₂, V₆O₁₃, Y₂O₃, ZrO₂); metal sulfides (MoS₂, PbS); Prussian blue; silicon; and composites (FeOx-doped mesoporous silicon, TiO₂-loaded Fe₃O₄, Prussian blue-ferritin, Prussian blue-MnO₂, Pt-MOF composites). A review of materials with enzyme-like materials and all listed references is incorporated here in its entirety (106). In acidic environments, such as the vagina, MnO₂ degrades to produce O₂ with high reactivity and specificity with and without H₂O₂.

The MnO₂ can be incorporated into the encapsulation device as nanoparticles, nanosheets, or coatings on nanoparticle carriers such as silicon or aluminum oxides.

In cases where insufficient H₂O₂ is naturally present in the vaginal fluids, H₂O₂ may be delivered through the use of a carrier, such as a liposome, for subsequent decomposition to form O₂, as described in (108). Instead of delivering H₂O₂ directly in the encapsulation device, H₂O₂ may be generated within the cell compartment or a separate, adjacent compartment separated from the cell compartment by an O₂ permeable or other suitable membrane. In one embodiment, a nanozyme core-shell system uses CaO₂ to generate O₂ following hydrolysis of CaO₂ to H₂O₂ and subsequent decomposition of H₂O₂ to O₂ using a catalyst as described above (109)}, incorporated herein in its entirety. Encapsulating CaO₂ with a MnO₂ nanosheet forms a structure capable of self-generating O₂ within the encapsulation device.

### Target Encapsulation System Specifications

The drug delivery devices (e.g., vaginal encapsulation devices) disclosed herein differ from other systems described in the art. For example, the device contains live cells that produce one (or more) pharmaceutically active substance(s) (e.g., agent(s)) over a period of time. The device geometry and dimensions do not restrict the amount of the agent(s) that can be delivered over the period of use, as the agent(s) is(are) produced over time by the cells contained in the device. In traditional systems described in the art, the agent(s) is(are) incorporated into the device as a depot of predetermined mass, and the period of use of devices known in the art is therefore restricted. For example, an IVR known in the art delivering 10 mg per day of an agent, and containing 300 mg of the agent cannot have a period of use beyond 30 days (10×30=300).

The amount of pharmaceutically active substance(s) (e.g., agent(s)) produced and released by the drug delivery devices (e.g., vaginal encapsulation devices) disclosed herein can be calculated as a pharmaceutically effective amount, where the devices of the present disclosure produce and release a pharmaceutically effective amount of one or more pharmaceutically active substances (e.g., agents). By “pharmaceutically effective”, it is meant an amount that is sufficient to effect the desired physiological or pharmacological change in subject. This amount will vary depending upon such factors as the potency of the particular pharmaceutically active substance, the efficiency of cellular production of the pharmaceutically active substance over time, the desired physiological or pharmacological effect, and the time span of the intended treatment. Those skilled in the pharmaceutical arts will be able to determine the pharmaceutically effective amount for any given pharmaceutically active substance in accordance with the standard procedure.

### Encapsulation System Fabrication

### Membrane Fabrication

Porous material or materials can be used in membrane fabrication, as described in detail above. In one embodiment, the membrane is formed from a porous membrane of polyurethane, silicone, or other suitable elastomeric material. Open cell foams and their production are known to those skilled in the art. Open cell foams may be produced using blowing agents, typically carbon dioxide or hydrogen gas, or a low-boiling liquid, present during the manufacturing process to form closed pores in the polymer, followed by a cell-opening step to break the seal between cells and form an interconnected porous structure through which diffusion may occur. An alternative embodiment employs a breath figure method to create an ordered porous polymer membrane (110). In this method, a hexagonal array of micrometric pores is obtained by water droplet condensation during fast solvent evaporation performed under a humid flow. Porous membranes may also be fabricated using porogen leaching methods, whereby a polymer is mixed with salt or other soluble particles of controlled size prior to casting, spin-coating, extrusion, or other processing into a desired shape. The polymer composite is then immersed in an appropriate solvent, and the porogen particles are leached out leaving structure with porosity controlled by the number and size of leached porogen particles. A preferred approach is to use water-soluble particles and water as the solvent for porogen leaching and removal. Highly porous scaffolds with porosity values up to 93% and average pore diameters up to 500 μm can be formed using this technique. A variant of this method is melt molding and involves filling a mold with polymer powder and a porogen and heating the mold above the glass-transition temperature of the polymer to form a scaffold. Following removal from the mold, the porogen is leached out to form a porous structure with independent control of morphology (from porogen) and shape (from mold).

A phase separation process can also be used to form porous membranes using methods known in the art (111). A second solvent is added to a polymer solution (quenching) and the mixture undergoes a phase separation to form a polymer-rich phase and a polymer-poor phase. The polymer-rich phase solidifies and the polymer poor phase is removed, leaving a highly porous polymer network, with the micro- and macro-structure controlled by parameters such as polymer concentration, temperature, and quenching rate. A similar approach is freeze drying, whereby a polymer solution is cooled to a frozen state, with solvent forming ice crystals and polymer aggregating in interstitial spaces. The solvent is removed by sublimation, resulting in an interconnected porous polymer structure. A final method for forming porous polymer membranes is using a stretching process to create an open-cell network (112).

Porous metal materials may be fabricated by traditional sintering processes known in the art (113). Loose powder or gravity sintering creates pores from the voids in the packed powder as grains join by a diffusional bonding process. Pore size and density is determined primarily by the morphology of the starting metal powder material and is difficult to control. Porogens may be used to create open-cell, interconnected metal foams of ca. 35-80% porosity with 100-600 μm pore size in a method analogous to those described herein for polymer foams. Porogens may include salts (e.g., NaCl, NaF, and NH₄HCO 3), organic materials (e.g., tapioca starch, urea), or other metals (e.g., magnesium). Porogens are removed to form pores thermally during sintering or in a post-sintering process, or by dissolution in a solvent. The high melting temperature (1310° C.) of Nitinol limits preparation methods of porous materials to powder metallurgy techniques. Materials can be prepared by sintering of Ni and Ti powders in predetermined ratios to form NiTi alloys during the sintering process. Alternatively, pre-alloyed NiTi powders may be sintered with or without additional porogens to form porous structures with controlled Ni:Ti ratios.

### Device Assembly

Open ends of the drug delivery device can be plugged with a pre-manufactured end plug to ensure a smooth end and a solid seal. Plugs may be sealed in the drug delivery device end using frictional force (for example, a rim and groove that lock together to form a seal); an adhesive; induction or laser welding, or another form of heat sealing that melts together the plug and drug delivery device end. In another embodiment, the ends are sealed without using a solid plug by one of a number of methods known to one skilled in the art, including but not limited to, heat-sealing, induction welding, laser welding, or sealing with an adhesive.

In some embodiments, one or more membranes may be mounted on the scaffold by ultrasonic welding. In further embodiments, an adhesive may be used at the region where the membranes adjoin the scaffold. The adhesive may be used to seal the junction between the membrane(s) and the scaffold, for instance to prevent leakage of fluids into or out of the chamber. The adhesive may also assist fixation of the membrane(s) to the scaffold, whether or not the membrane(s) are also welded ultrasonically to the frame. An adhesive may be used around part or the whole of the periphery of the chamber; e.g., along a part or a whole of a perimeter of the first and/or second membranes, and/or along a part or a whole of an internal surface of the first and/or second scaffold elements. Any suitable biocompatible adhesive may be used. In one embodiment, a photopolymerizable adhesive is used.

### Additive Manufacturing of the Encapsulation Device

Additive manufacturing—colloquially referred to as 3D printing technology in the art—is one of the fastest growing applications for the fabrication of plastics. Components that make up the drug delivery device can be fabricated by additive techniques that allow for complex, non-symmetrical three-dimensional structures to be obtained using 3D printing devices and other methods known to those skilled in the art (114, 115), incorporated herein by reference. There are currently three principal methods for additive manufacturing: stereolithography (SLA), selective laser sintering (SLS), and fused deposition modeling (FDM).

The SLA process requires a liquid plastic resin, a photopolymer, which is then cured by an ultraviolet (UV) laser. The SLA machine requires an excess amount of photopolymer to complete the print, and a common g-code format may be used to translate a CAD model into assembly instructions for the printer. An SLA machine typically stores the excess photopolymer in a tank below the print bed, and as the print process continues, the bed is lowered into the tank, curing consecutive layers along the way. Due to the smaller cross-sectional area of the laser, SLA is considered one of the slower additive fabrication methods, as small parts may take hours or even days to complete. Additionally, the material costs are relatively higher, due to the proprietary nature and limited availability of the photopolymers. In one embodiment of the disclosure, one or more components of the drug delivery device is fabricated by an SLA process.

The SLS process is similar to SLA, forming parts layer by layer through use of a high energy pulsed laser. In SLS, however, the process starts with a tank full of bulk material in powder form. As the print continues, the bed lowers itself for each new layer, advantageously supporting overhangs of upper layers with the excess bulk powder not used in forming the lower layers. To facilitate processing, the bulk material is typically heated to just under its transition temperature to allow for faster particle fusion and print moves (116). In one embodiment of the disclosure, one or more components of the drug delivery device is fabricated by an SLS process.

Rather than using a laser to form polymers or sinter particles together, FDM works by extruding and laying down consecutive layers of materials at high temperature from polymer melts, allowing adjacent layers to cool and bond together before the next layer is deposited. In the most common FDM approach, fused fiber fabrication (FFF), polymer in the form of a filament is continuously fed into a heated print head print whereby it melts and is deposited onto the print surface. The print head moves in a horizontal plane to deposit polymer in a single layer, and either the print head or printing platform moves along the vertical axis to begin a new layer. A second FDM approach uses a print head design based on a traditional single-screw extruder to melt polymer granulate (powders, flakes, or pellets) and force the polymer melt through a nozzle whereby it is deposited on the print surface similar to FFF. This approach allows the use of standard polymer materials in their granulated form without the requirement of first fabricating filaments through a separate extrusion step. In one embodiment of the disclosure, one or more components of the drug delivery device is fabricated by an FDM and/or FFF process.

In another embodiment, Arburg Plastic Freeforming (APF) (117) is the additive manufacturing technique used in drug delivery device fabrication. In this embodiment, a plasticizing cylinder with a single screw is used to produce a homogeneous polymer melt similarly to the process for thermoplastic injection molding. The polymer melt is fed under pressure from the screw cylinder to a piezoelectrically actuated deposition nozzle. The nozzle discharges individual polymer droplets of controlled size in a pre-calculated position, building up each layer of the 3-dimensional polymer print from fused droplets. The screw and nozzle assembly are fixed in location, and the build platform holding the printed part is moved along three axes to control droplet deposition position. The droplets bond together on cooling to form a solid part. This technique can operate at elevated temperatures (ca. 300° C.) and pressures (ca. 400 bar). One advantage of the APF method is that it is directly compatible with many of the processes used in injection molding and extrusion (e.g., granulated polymer feedstocks, no organic solvents).

In another embodiment, droplet deposition modelling (DDM) is used as the additive manufacturing technique by producing discrete streams of material during deposition, known for inkjet systems.

A preferred method of additive manufacturing that avoids sequential layer deposition to form the three-dimensional structure is to use continuous liquid interface production (CLIP), a technique recently developed by Carbon3D. In CLIP, three dimensional objects are built from a fast, continuous flow of liquid resin that is continuously polymerized to form a monolithic structure with the desired geometry using UV light under controlled oxygen conditions. The CLIP process is capable of producing solid parts that are drawn out of the resin at rates of hundreds of mm per hour. Drug delivery device scaffolds containing complex geometries may be formed using CLIP from a variety of materials including polyurethane and silicone.

### Use and Applications of the Drug Delivery (Encapsulation) Device

### Use and Use Restrictions

The drug delivery devices (e.g., vaginal encapsulation devices) disclosed herein are placed in the vagina during use and delivers one or more agents to the vaginal mucosa. In one embodiment, the agents are active locally and/or in the female reproductive tract. In another embodiment, the agents are active locally in the vagina and in the rectum. In another embodiment, the agents are absorbed systemically and act systemically. In yet another embodiment, the agents are active in a combination of the above pharmacologic compartments.

### Vaginal Immunization

Many pathogens initiate infections via mucosal surfaces, including infectious agents that generally remain localized (e.g., Neisseria gonorrhea) or disseminate systemically (e.g., HIV). While mucosal immunity remains incompletely understood, it appears that there is compartmentalization of the immune system between mucosal and systemic immunity. Additionally, there may be some degree of compartmentalization between different mucosal compartments, such as genitourinary and respiratory locations. Increasing evidence indicates that there are “tissue resident memory T cells” that do not circulate, for example. It is therefore recognized that systemic immunization may not consistently elicit effective mucosal immune responses at key sites of infection.

In general, however, mucosal-targeted vaccinations have met with limited success in generating locally protective mucosal immunity against pathogens. The major exceptions use vaccines that are live-attenuated pathogens (e.g., oral polio and cholera vaccines; and nasal influenza vaccine), indicating that a key requirement is sustained antigen exposure at mucosal sites. Although there has been some limited success in attempts at mucosal vaccination, such as described in (118) incorporated herein by reference, this requirement is supported by many failed oral and vaginal vaccination trials using protein subunit antigens.

The disclosure offers a novel and non-obvious approach to providing prolonged, selectively intermittent antigenic vaginal mucosal exposure alternative to the use of live-attenuated pathogens, which may pose safety and/or development challenges (e.g., Neisseria gonorrhea, Chlamydia trachomatis, Herpes Simplex Virus, Treponema pallidum). Vaginal encapsulation of cells that produce antigens of interest for prolonged periods of time can provide immune stimulatory exposure for days or weeks, mimicking the exposure offered by a live-attenuated pathogen. This would allow elicitation of vaginal mucosal immunity, focusing responses at the key site of vaginal pathogens. Exposure to said antigens also can be stopped at any moment simply by removing the vaginal device, a major advantage of the disclosed devices.

### Exemplary Applications

Provided herein are methods of treating or preventing diseases and disorders in a subject in need thereof, comprising administering to the subject a drug delivery device disclosed herein. A primary non-limiting purpose of the drug delivery devices (e.g., vaginal encapsulation devices) described herein is to deliver one or more agents to the vaginal mucosa for the purposes of treating, preventing, reducing the likelihood of having, reducing the severity of and/or slowing the progression of a medical condition in a subject. The application can be local (La, vaginal, female reproductive tract, rectum) or systemic, or a combination.

An advantage of the disclosed drug delivery devices is increased patient compliance by reducing problems in adherence to treatment and prevention associated with more frequent dosing regimens. Consequently, the drug delivery devices of the disclosure can be used for a plurality of applications. Illustrative, non-restrictive examples of such applications are provided below in summary form. Based on these examples, one skilled in the art could adapt the disclosed technology to other applications.

In some embodiments, the methods disclosed herein comprise treating or preventing a disease or disorder in a subject in need thereof, comprising administering to the subject a drug delivery device disclosed herein. In some embodiments, the disease or disorder is selected from infectious diseases, transplants-graft rejection, physiological and pathophysiological disorders, diabetes mellitus, allergies and hypersensitivities, autoimmune disorders, oncological disorders, hematologic diseases, musculoskeletal diseases and disorders, psychological and neurologic disorders, and genetic diseases and disorders; the methods also comprise hormonal therapies, and veterinary applications. In some embodiments, the disease or disorder is a sexually transmitted infection (STI). In some embodiments, the sexually transmitted infection is Neisseria gonorrhea, Chlamydia trachomatis, Herpes Simplex Virus, Treponema pallidum, or Human Immunodeficiency Virus (HIV) infection. In some embodiments, the methods disclosed herein comprise methods of contraception comprising administering to a subject in need thereof a drug delivery device disclosed herein.

Infectious Diseases, including multiple, overlapping infections: sexually transmitted infections (STIs), including but not limited to prevention or treatment, both active and chronic active, with one or more suitable antimicrobial agents delivered from the device. Illustrative, but not limiting examples of STIs include: gonorrhea, chlamydia, lymphogranuloma venereum, syphilis, including multidrug-resistant (MDR) organisms, hepatitis C virus, and herpes simplex virus, and HIV prevention using one or more one or more suitable antiretroviral agents, including biologics, and/or one or more vaccines and/or adjuvants delivered from the device; and treatment, using one or more suitable antiretroviral agents, including biologics, delivered from the device; bacterial vaginosis (BV) prevention or treatment, both active and chronic active, with one or more suitable agents delivered from the device; hepatitis B virus (HBV) prevention or treatment, both active and chronic active, with one or more suitable antiviral agents delivered from the device; herpes simplex virus (HSV) and varicella-zoster virus (shingles) Zoster/Shingles, prevention or treatment, both active and chronic active, with one or more suitable antiviral agents delivered from the device; cytomegalovirus (CMV) and congenital CMV infection, prevention or treatment, both active and chronic active, with one or more suitable antiviral agents delivered from the device; malaria, prevention or treatment, both active and chronic active, with one or more suitable antimicrobial agents delivered from the device; tuberculosis, including multidrug-resistant (MDR) and extensively drug-resistant (XDR) tuberculosis, prevention or treatment, both active and chronic active, with one or more suitable antibacterial agents delivered from the device; and acne, treatment or management with one or more suitable agents delivered from the device.

Transplants—Graft Rejection: chronic immune-suppressive post-transplant therapy with one or more suitable agents delivered from the device.

Hormonal Therapy: Contraception, including estrogens and progestins, with one or more suitable agents delivered from the device; hormone replacement, with one or more suitable agents delivered from the device, testosterone replacement, with one or more suitable agents delivered from the device; thyroid replacement/blockers, with one or more suitable agents delivered from the device; steroid and other treatments for adrenal insufficiency (Addison's disease) and other chronic deficiencies (or excess) from pituitary as well as pituitary adrenal axis; hormonal treatment to regulate triglycerides (TGs) using one or more suitable agents delivered from the device; and chronic pharmacologic support for all transgender individuals (all stages from cis-trans), using one or more suitable agents delivered from the device.

Physiology and Pathophysiology: gastrointestinal (GI) applications, with one or more suitable agents delivered from the device, including, but not limited to the treatment/management of diarrhea, pancreatic insufficiency, cirrhosis, fibrosis in all organs; GI organs-related parasitic diseases, gastroesophageal reflux disease (GERD); cardiovascular applications, with one or more suitable agents delivered from the device, including, but not limited to the treatment/management of hypertension (HTN) using, for example, statins or equivalent, cerebral/peripheral vascular disease, stroke/emboli/arrhythmias/deep venous thrombosis (DVT) using, for example anticoagulants and anti-atherosclerotic cardiovascular disease (ASCVD) medications, and congestive heart failure (CHF) using for example β-blockers, ACE inhibitors, and angiotensin receptor blockers; pulmonary applications, with one or more suitable agents delivered from the device, including, but not limited to the treatment/management of sleep apnea, asthma, longer-term pneumonia treatment, pulmonary HTN, fibrosis, and pneumonitis; bone applications, with one or more suitable agents delivered from the device, including, but not limited to the treatment/management of chronic pain (joints as well as bone including sternal), osteomyelitis, osteopenia, cancer, idiopathic chronic pain, and gout; urology applications, with one or more suitable agents delivered from the device, including, but not limited to the treatment/management of benign prostatic hyperplasia (BPH), bladder cancer, chronic infection (entire urologic system), chronic cystitis, prostatitis; ophthalmology applications, with one or more suitable agents delivered from the device, including, but not limited to the treatment/management of glaucoma, ocular infections; cholesterol management, with one or more suitable agents delivered from the device; and metabolic applications, with one or more suitable agents delivered from the device, including, but not limited to the treatment/management of weight gain, weight loss, obesity, malnutrition (replacement), osteopenia, Vitamin deficiency (B vitamins/D), folate, and smoking/drug reduction/cessation.

Diabetes mellitus: treatment and management of diabetes (type 1 and 2), with one or more suitable agents (including peptide drugs) delivered from the device,

Allergies and Hypersensitivities, with “desensitization”, often need low-dose repeated exposure: TYPES: Type I (IgE mediated reactions), Type II (antibody mediated cytotoxicity reactions), Type III (immune complex-mediated reactions), and Type IV for delayed type hypersensitivity (119), with one or more suitable agents delivered from the device; hypersensitivity reactions (HSRs), with one or more suitable agents delivered from the device; antibiotics, biologics (drug and antibody portion), chemotherapy (e.g., platins), progesterone, as well as other treatments known and described in (119), with one or more suitable agents delivered from the device; food allergies (e.g., nuts, shellfish) with one or more suitable agents delivered from the device; and allergy medication dosing with one or more suitable agents delivered from the device, as an alternative to allergy shots, recommended for people with severe allergy symptoms who do not respond to usual medications; for people who have significant medication side effects from their medications; for people who find their lives disrupted by allergies/insect stings; or people for whom allergies might become life threatening: anaphylaxis.

Autoimmune Disorders, often classified as chronic inflammatory disorders: treatment and management of Crohn's disease and ulcerative colitis, with one or more suitable agents (e.g., biologics) delivered from the device; rheumatoid arthritis (RA) treatment and management with one or more suitable agents (e.g., biologics) delivered from the device; multiple sclerosis (MS) treatment and management with one or more suitable agents (e.g., biologics) delivered from the device; psoriasis treatment and management with one or more suitable agents (e.g., biologics) delivered from the device; lupus treatment and management, including but not limited to systemic lupus erythematosus, with one or more suitable agents (e.g., biologics) delivered from the device; diabetes mellitus, Type 1 (vide supra), treatment and management with one or more suitable agents (e.g., biologics); Addison's disease treatment and management with one or more suitable agents (e.g., biologics); Graves' disease treatment and management with one or more suitable agents (e.g., biologics); and autoimmune thyroiditis treatment and management with one or more suitable agents (e.g., biologics) delivered from the device.

Oncology: chemotherapy and targeted therapy (e.g., Ig) chronic or sub-chronic cancer management with one or more suitable agents delivered from the device.

Hematologic Diseases: treatment/management of Hemophilia A with one or more suitable agents (e.g., Factor VIII orthologs) delivered from the device; administration of anticoagulants and/or antiplatelet therapy with one or more suitable agents delivered from the device; treatment/management of leukemia/lymphoma and bone marrow transplant (MBT) therapies with one or more suitable agents delivered from the device; iron replacement therapy with one or more suitable agents delivered from the device; and fibroproliferative disorders required blockade.

Musculoskeletal Applications: delivery of one or more anti-inflammatory agents (e.g., NSAIDS) from the device; delivery of low-dose prednisone from the device; opioids addiction/pain management with one or more suitable agents delivered from the device; and hypertrophic fibrosis/scar tissue.

Psychological and Neurologic Disorders: treatment and management of depression with one or more suitable agents delivered from the device; treatment and management of schizophrenia, and related, with one or more suitable agents delivered from the device; treatment and management of bipolar disorders with one or more suitable agents delivered from the device; treatment and management of dysthymic disorders with one or more suitable agents delivered from the device; treatment and management of seizure control with one or more suitable agents delivered from the device; treatment and management of ADD/ADHD and hyperactivity disorders with one or more suitable agents delivered from the device; treatment and management of behavioral/emotional secondary to early-onset (child/adolescent), substance use, physical, sexual, emotional abuse, PTSD, and anxiety with one or more suitable agents delivered from the device; treatment and management of Parkinson's disease with one or more suitable agents delivered from the device; and treatment and management of Alzheimer's disease with one or more suitable agents delivered from the device.

Genetic Diseases: treatment of congenital genetic deficiency diseases, including genetic excess diseases, with one or more suitable agents delivered from the device; treatment of primary immunodeficiencies (e.g., agammaglobulinemia, secretory IgA deficiency, sIgA deficiency) with one or more suitable agents delivered from the device; severe combined immunodeficiency (SCID) treated SCID with one or more suitable agents delivered from the device, including, but not limited to enzyme replacement therapy (ERT) with pegylated bovine ADA (PEG-ADA); muscular dystrophy treated and managed with one or more suitable agents delivered from the device; treatment or management of Duchenne's disease with one or more suitable agents (e.g., eteplirsen) delivered from the device; treatment or management of Pompe's disease with one or more suitable agents delivered from the device, including ERT such as intravenous administration of recombinant human acid α-glucosidase; and treatment or management of Gaucher disease with one or more suitable agents delivered from the device, including ERT.

Veterinary Applications involving all mammals, including, but not limited to dogs, cats, horses, pigs, sheep, goats, and cows.

In one embodiment of the disclosure, the drug delivery devices (e.g., vaginal encapsulation devices) serve multiple purposes, where more than one application is targeted simultaneously. An example of such a multipurpose drug delivery device (e.g., vaginal encapsulation device) involves the prevention of HIV infection, with the delivery of one or more microbicidal agents, and contraception, with the delivery of one or more contraceptive agents (e.g., anti-sperm antibodies).

In one embodiment of the disclosure, the drug delivery devices (e.g., vaginal encapsulation devices) serve multiple purposes, where one or more applications involve the producing and release of one or more agents by the cells. In some embodiments, a structural function is performed by the device, for example a pessary-shaped device to help support the bladder, vagina, uterus, and/or rectum. The post-treatment application of the drug delivery devices (e.g., vaginal encapsulation devices)—for example, but not limited to, following: surgery, chemotherapy, radiation, motor-vehicle accidents, MVA, war-related injuries—can include embodiments where the device is used as a vaginal structural support.

In one embodiment, the cells contained in the device produce and release nucleic acid polymers, including messenger ribonucleic acids (mRNAs)—synthetic or natural—to stimulate the in vivo expression of one or more proteins (55), such as antibodies (56) and vaccine adjuvants (57) in the vaginal mucosa (i.e., by vaginal epithelial cells or other cell types present in the vaginal mucosa), external to the device.

Cellular implants are uncommon and none are intended for vaginal use. Aebischer and colleagues have reported on a number of applications involving cellular implants, including intrathecal delivery of CNTF (120), subcutaneous gene therapy for the regulated delivery of therapeutic proteins (121), striatal delivery of GDNF (122), and subcutaneous passive immunization (74).

The cellular encapsulation devices described in the art are intended to be implanted into a sterile body compartment, mostly subcutaneously. A significant benefit of this approach is that the highly vascular environment leads to the development of dense neovascularization around the device, thereby nourishing the cells (74). This does not occur with a vaginal encapsulation device. In addition, the vaginal milieu is colonized by polymicrobial communities that produce a wide range of substances that can be deleterious to the encapsulated cells, including lactic acid that can depress the vaginal pH to 3.8, or less (123). In addition, the vagina is microaerophilic; a high carbon dioxide, low oxygen environment, further complicating the growth of certain cell lines. In other instances, one or more compounds produced vaginally may stimulate or enhance the production of the target agents by the encapsulated cells. The drug delivery devices disclosed herein contravene these disadvantages.

It is known that the release kinetics of the agent(s) from sustained release drug delivery systems (SRDDSs) is controlled by osmosis and/or by diffusion, and is usually zero order, pseudo-zero order, or first order. By contrast, the release of agent(s) from cells encapsulated in the devices disclosed herein is controlled by numerous, complex factors, such as the concentration or nutrients, the metabolic state of the of the cells, the expression of intracellular enzymes and proteins, including transporter proteins, and the permeability (active and/or passive) of the agent(s) across one or more biological membranes. Importantly, the agent(s) is(are) produced by the living cells over time, while a SRDDS contains the entire API payload at administration.

### EQUIVALENTS

The present disclosure is not to be limited in terms of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

As a person skilled in the art would readily know many changes can be made to the preferred embodiments of the disclosure without departing from the scope thereof. It is intended that all matter contained herein be considered illustrative of the disclosure and not in a limiting sense.

While various aspects and embodiments have been disclosed herein, other aspects and embodiments will be apparent to those skilled in the art. All references cited herein are incorporated by reference in their entireties.

### EXAMPLES

### Example 1—Wild Type Bacteria (Single Strain) for Vaginal Delivery of Lactic Acid

Polyurethane IVR, 310 (FIG. 3), holds two cellular encapsulation devices, 321 (FIG. 3). A lyophilized powder consisting of Lactobacillus crispatus cells (10¹⁰ CFU per chamber), nutrients (D-glucose-glycogen, 2:1 w/w), and a PVA-sodium alginate blend (2:1 w/w) is pre-weighed and filled into both lobe-shaped chambers. A low density, permeable ePTFE membrane, 322, is placed over the filled chamber, followed by protective nylon mesh, 323. Sealing ring, 324, is placed over the mesh and the device is sealed by ultrasonic welding. Device assembly is carried out in a laminar flow hood using aseptic conditions. The exterior surface of the completed device is cleaned with a cleanroom iso-propanol wipe and packaged individually in a moisture/O₂ barrier pouch that is subsequently sealed. The device is stored at room temperature prior to use.

The bacterial strain was chosen specifically because it is an efficient producer of L-lactic acid, a short chain fatty acid with known microbicidal properties. The IVR described here can be used to prevent or treat bacterial vaginosis, as well as to prevent sexually transmitted infections, including HIV-1.

### Example 2—Wild Type, Blended Bacteria (Multiple Strains) for Vaginal Delivery of Lactic Acid

The same approach as in EXAMPLE 1 is used, but the lyophilized powder is made up of multiple vaginal bacterial species, mixed to a final concentration of 10¹⁰ CFU per chamber. Exemplary vaginal bacterial species include: Lactobacillus crispatus, L. gasseri, L. jensenii, L. rhamnosus, and L. iners Here, nylon mesh, 323, and sealing ring, 324, are combined into a single protective cover equipped with slots that seals over membrane, 322.

### Example 3—Wild Type, Separate Bacteria (Multiple Strains) for Vaginal Delivery of Lactic Acid

As in Example 2, a plurality of bacterial species is used, but the lyophilized powder of each species along with the excipients described in Example 1 is contained individually (i.e., all species kept separate) in dedicated chambers using IVR design, 106 (FIG. 1). Release of lactic acid produced by the bacteria is achieved through a porous, semipermeable Teflon™ membrane. The exterior surface of the completed device is cleaned with a cleanroom iso-propanol wipe and packaged individually in a moisture/O₂ barrier pouch that is subsequently sealed. The device is stored at room temperature prior to use.

### Example 4—Recombinant Bacteria Producing Microbicidal Agents

The same approach as in EXAMPLE 1 is used, but the lyophilized powder is made up of a recombinant vaginal bacterial species, at a final concentration of 10¹⁰ CFU per chamber. A non-limiting example of a recombinant bacterial species includes genetically modified Lactobacillus spp., such as L. jensenii modified to express broadly neutralizing antibodies against epitopes on the HIV-1 envelope. A non-limiting example of a relevant organism has been reported by Chang et al. (124), incorporated herein in its entirety by reference.

### Example 5—Recombinant Yeast Cells Expressing Proteins

Poly(ethylene-co-vinyl acetate) (EVA) IVR, 600 (FIG. 6), comprises a lower element, 621, comprising one or more chambers to contain cells. The chambers are covered by an upper element, 622, that forms the upper portion of the cell chambers and holds a permeable membrane, 623, on top to retain cells in the chamber. In some cases, the membrane is retained in 622 by a ring, 624. Elements 621, 622, and 624 are made of EVA in this example. Porous, semipermeable membrane 273 consists of hydrophilic polypropylene.

The chamber, 621, is filled with a lyophilized powder consisting of recombinant, non-pathogenic Candida albicans cells of vaginal origin (10⁸ CFU), nutrients (D-glucose-peptone, 2:1 w/w), and hyaluronic acid-PLGA-co-PEG (2:1 w/w) is pre-weighed and filled into the chamber and covered with a PTFE nanofiber mat. Assembly of the components is carried out in a laminar flow hood using aseptic conditions, and sealed by ultrasonic welding. The exterior surface of the completed device is cleaned with a cleanroom iso-propanol wipe and packaged individually in a moisture/O₂ barrier pouch that is subsequently sealed. The device is stored at room temperature prior to use.

The recombinant yeast cells can be engineered to express a range of proteins. These systems represent non-limiting embodiments of EXAMPLE 5. In one non-limiting example of the antiviral protein belongs to the lectin family, such as griffithsin (GRFT), cyanovirin-N (CV-N) and scytovirin (SVN) (59), incorporated herein in its entirety. In another nonlimiting example, the antiviral agent produced by the recombinant cells consists of a broadly neutralizing antibody (bNAb), such as VRC01 that possesses activity against various HIV-1 isolates (60). Other, next generation bNAbs that are more potent against HIV-1 or neutralize other viruses such as HSV or HPV, or bacteria, such as multidrug-resistant Neisseria gonorrhoeae, such as described in the art, incorporated herein by reference in its entirety (61-64). In another embodiment, the cells produce a nonhormonal contraceptive consisting of multivalent IgGs with high agglutination potencies for trapping vigorously motile sperm (65, 66).

### Example 6—Recombinant Yeast Cells Expressing Proteins Using Separated Chambers

The system described under EXAMPLE 5 is modified such that only one terminal chamber for each of the three chambers in close proximity to one another (FIG. 6) is filled with the cells and excipients. In other words, two of the six chambers are filled with cells. The two sets of three cells are separated by resorbable walls made up of PLGA. The two sets of two chambers that do not contain cells are filled with nutrients, growth factors, and vitamins and the polypropylene membranes are replaced with EVA membranes. In this example, the compounds produced by the cells are only released through the polypropylene membranes covering the first two chambers, one in each set of three, as the EVA membranes sealing the nutrient chambers are blocking the release of compounds. As the PLGA separations between chambers erode, the growing cells are able to access additional nutrients, thereby extending their vitality and the duration of use of the vaginal device.

### Example 7—Recombinant Mammalian Cells Expressing Peptides

IVR scaffold, 800 (FIG. 8), is made out of EVA and comprises an unmedicated component used to hold two cassette-style cellular chambers, 900 (FIG. 9). The chamber (FIG. 9) comprises an outer sealing ring, 903, that forms a seal with the membrane, 904, and rib structures, 905, that support the skin membrane and define compartments within the chamber. A non-porous sealing disk, 901, closes the cassette. The porous, semipermeable membrane, 904, consists of cellulose acetate, molecular weight cutoff (MWCO) 6-8 kDa.

Recombinant mammalian cells, in the form of a pellet collected by centrifugation, are loaded into the chamber, 902, along with a biomimetic poly(ethylene glycol) gel. In addition, nutrients, growth factors/hormones, O₂-generating agents, and pH buffering agents (in-capsule target pH=7.2) all are formulated as sustained-release, PLGA-coated beads. The beads are mixed with the cellular hydrogel suspension, and the devices are assembled and sealed by ultrasonic welding in a laminar flow hood using aseptic conditions. The exterior surface of the completed device is cleaned with a cleanroom iso-propanol wipe and packaged individually in a moisture/O₂ barrier pouch that is subsequently sealed. The device is stored at 4° C. prior to use.

The recombinant mammalian cells, spontaneously immortalized C8-B4 mouse microglial cell line, can be engineered to express a range of pharmaceutically active peptides. Another example of mouse cells that can be genetically modified to meet the purposes of the disclosed invention includes C2C12 cells. In another example, the genetically modified cells consist of Chinese hamster ovary (CHO) cells. These systems represent non-limiting embodiments of EXAMPLE 7. In non-limiting examples, the pharmaceutically active peptide includes: antiviral fusion inhibitors such as enfuvirtide, and derivatives thereof; broad antiviral peptides such as described by Cheng et al. (58), incorporated herein its entirety; peptides known in the art to treat or manage diabetes mellitus including, but not limited to exenatide, octreotide, goserelin, or derivatives and pharmaceutically acceptable salts thereof; and the GnRH agonist leuprolide, or derivatives and pharmaceutically acceptable salts thereof, used in the management and treatment of prostate cancer, endometriosis, uterine fibroids, precocious puberty, and other sex hormone-related conditions.

### Example 8—Recombinant Mammalian Cells Expressing Proteins

IVR scaffold, 800 (FIG. 14), is made out of polyurethane and comprises an unmedicated component used to hold two cassette-style cellular chambers, 900 (FIG. 9). The chamber (FIG. 9) comprises an outer sealing ring, 903, that forms a seal with the membrane, 904, and rib structures, 905, that support the skin membrane and define compartments within the reservoir. A non-porous sealing disk, 901, closes the cassette. The porous, semipermeable membrane, 904, consists of hydrophilic polypropylene.

The recombinant mammalian cells, in the form of a pellet collected by centrifugation, are loaded into the chamber, 902, along with a biomimetic poly(ethylene glycol) gel. In addition, nutrients, growth factors/hormones, O₂-generating agents, and pH buffering agents (in-capsule target pH=7.2) all are formulated as sustained-release, PLGA-coated beads. The beads are mixed with the cellular hydrogel suspension, and the devices are assembled and sealed by ultrasonic welding in a laminar flow hood using aseptic conditions. The exterior surface of the completed device is cleaned with a cleanroom iso-propanol wipe and packaged individually in a moisture/O₂ barrier pouch that is subsequently sealed. The device is stored at 4° C. prior to use.

In this example, the mammalian cells consist of recombinant human embryonic kidney 293 (HEK-293) cells, expressing one of the proteins described under EXAMPLE 5. The 293LTV cell line, derived from HEK-293, also can be used to produce the recombinant cells.

### Example 9—Recombinant Mammalian Cells Expressing Proteins Using Separated Chambers

IVR scaffold, 800 (FIG. 8), is made out of polyurethane and comprises an unmedicated component used to hold two cassette-style cellular chambers, 900 (FIG. 9). The chamber (FIG. 9) comprises an outer sealing ring, 903, that forms a seal with the membrane, 904. In this example, rib structures, 905, that support the skin membrane and define compartments within the reservoir are made of resorbable PLGA. A PLGA non-porous sealing disk, 901, closes the cassette. The porous, semipermeable membrane, 904, consists of hydrophilic polypropylene.

The recombinant mammalian cells, in the form of a pellet collected by centrifugation, are loaded into one compartment of chamber, 900, along with a biomimetic poly(ethylene glycol) gel, nutrients, buffers, and compounds needed to maintain cell vitality. The remaining chambers within the capsule are loaded with biomimetic poly(ethylene glycol) gel containing nutrients, growth factors/hormones, O₂-generating agents, and pH buffering agents (in-capsule target pH=7.2) all formulated as sustained-release, PLGA-coated beads. The beads are mixed with the cellular hydrogel suspension. The devices are assembled and sealed by ultrasonic welding in a laminar flow hood using aseptic conditions. The exterior surface of the completed device is cleaned with a cleanroom iso-propanol wipe and packaged individually in a moisture/O₂ barrier pouch that is subsequently sealed. The device is stored at 4° C. prior to use.

In this example, the cellular encapsulation device represents a personalized medicine application as the cells are collected from the patient. Cervicovaginal epithelial cells are harvested, immortalized, and transduced using the appropriate vectors using methods known in the art. The cells are modified to express one of the proteins described under EXAMPLE 5. Individual clones that meet the requirements of the cellular vaginal device are isolated and grown in culture.

### Example 10—Recombinant Mammalian Cells Expressing Proteins in a Microfluidic Chamber

IVR scaffold, 800 (FIG. 8), is made out of EVA and comprises an unmedicated component used to hold two cassette-style cellular chambers, 900 (FIG. 9). In this example, the two chambers consist of microfluidic chambers, as described above. Here, the microfluidic chips are made of PDMS and employ osmotic pumping to drive fluid flow. The porous, semipermeable membrane consists of hydrophilic polypropylene.

The recombinant mammalian cells, in the form of a pellet collected by centrifugation, are loaded into the sterile microfluidic chip along with a biomimetic poly(ethylene glycol) gel, nutrients, buffers, and compounds needed to maintain cell vitality. The devices are assembled and sealed by ultrasonic welding in a laminar flow hood using aseptic conditions. The exterior surface of the completed device is cleaned with a cleanroom iso-propanol wipe and packaged individually in a moisture/O₂ barrier pouch that is subsequently sealed. The device is stored at 4° C. prior to use.

In this example, the employed mammalian cells consist of 293LTV cells as described under EXAMPLE 7.

### Example 11—Recombinant Mammalian Cells for Vaginal Mucosal Immunization Against HSV

IVR scaffold, 800 (FIG. 8), is made out of polyurethane and comprises an unmedicated component. IVR scaffold, 800 (FIG. 8), is made out of polyurethane and comprises an unmedicated component used to hold two cassette-style cellular chambers, 900 (FIG. 9). The reservoir (FIG. 9) comprises an outer sealing ring, 903, that forms a seal with the membrane, 904, and rib structures, 905, that support the skin membrane and define compartments within the chamber. A non-porous sealing disk, 901, closes the cassette. The porous, semipermeable membrane, 904, consists of hydrophilic polypropylene.

Recombinant mammalian cells, in the form of a pellet collected by centrifugation, are loaded into the chamber, 902, along with a biomimetic poly(ethylene glycol) gel. In addition, nutrients, growth factors/hormones, O₂-generating agents, and pH buffering agents (in-capsule target pH=7.2) all are formulated as sustained-release, PLGA-coated beads. The beads are mixed with the cellular hydrogel suspension, and the devices are assembled and sealed by ultrasonic welding in a laminar flow hood using aseptic conditions. The exterior surface of the completed device is cleaned with a cleanroom iso-propanol wipe and packaged individually in a moisture/O₂ barrier pouch that is subsequently sealed. The device is stored at 4° C. prior to use.

In this example, intravaginal immunization against HSV is achieved with recombinant viral subunit protein gB plus in tandem with CpG oligodeoxynucleotides (ODNs) as adjuvant. This system is known in the art to induce high levels of gB-specific IgA and IgG in vaginal secretions and serum (125), incorporated herein in its entirety. Recombinant Chinese hamster ovary (CHO) cells expressing viral subunit protein gB plus are used in one of the two reservoirs, while CHO cells expressing ODNs are used in the other.

### Example 12—In Vitro Fluorescent Protein Release from Encapsulated Recombinant Human Monocytes

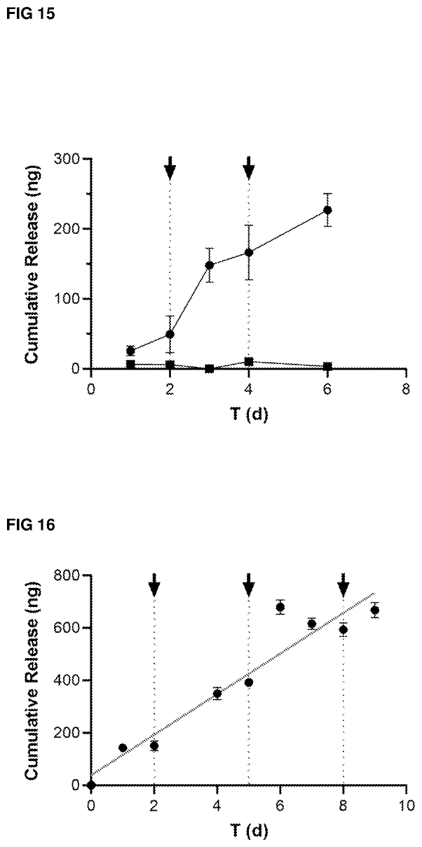

THP1-Dual™ cells (InvivoGen, San Diego, CA) were used as the model system for in vitro studies. The cell line is derived from the human THP-1 monocyte cell line by stable integration of two inducible reporter constructs. One of these constructs results in the expression of a secreted luciferase enzyme (62 kDa), under the control of a 2′3′-cGAMP inducer (1-3 μg mL⁻¹). The cell line was established in culture (no encapsulation) to verify the induction of luciferase secretion as follows. The cells were seeded at 1×10⁶ cells mL⁻¹ in 6-well tissue culture plates at 37° C., 5% CO₂, using the following growth medium: RPMI 1640, 2 mM L-glutamine, 25 mM HEPES, 10% heat-inactivated fetal bovine serum, 100 μg mL⁻¹ Normocin™, and Pen-Strep (100 U mL⁻¹-100 μg mL⁻¹) according to the manufacturer's instructions. Luciferase induction was carried out every 48 h (2′3′-cGAMP, 1 μg mL⁻¹) as part of the media exchange. Aliquots (50 μL) were removed daily to measure luciferase activity using the Quanti-Luc assay according to the manufacturer's instructions. FIG. 15 shows the successful induction of luciferase in this system over 6 days and multiple media changes. No fluorescence was observed when the inducer was omitted from the media.

A cassette style drug delivery device (FIG. 10) comprising a propylene body, 1011, and sealing ring and a silicone chamber disk, 1015, was loaded with a suspension of THP1-Dual™ cells (1×10⁶ cells mL⁻¹, 300 μL) and the chamber sealed using an ePTFE membrane, 1010, (25 mm diameter, 254 μm thickness, unsintered; Zeus Industrial Products, Inc., Orangeburg, SC) under aseptic conditions. The loaded cassettes were immersed in growth medium (35 mL), one device per vessel, and incubated at 37° C., 5% CO₂. Media exchanges containing the inducer (2′3′-cGAMP, 1 μg mL⁻¹) were carried out at predetermined timepoints. The devices were removed after 9 days of in vitro evaluation and the viability of the encapsulated cells was measured, as well as the growth rate. Cellular escape from the drug delivery devices was measured by microscopic examination and culture of the surrounding media.

Linear release of luciferase from the drug delivery device was observed for 9 days (FIG. 16), and likely would have continued if the experiment had not been stopped. Unexpectedly, the cell doubling time in the device (ca. 7.7 d) was considerably slower compared to free culture (1.1 d), while the rate of luciferase secretion was higher for the encapsulated cells (FIGS. 15 and 16). These results are counter-intuitive, as the encapsulated cells reproducing 7 times slower would have been expected to secrete luciferase at a lower rate relative to free culture. It was also surprising that despite the confines of encapsulation, cell viability remained high (74% at Day 9 compared to 98% at device loading).

All references are incorporated herein in their entirety.

### REFERENCES CITED

1. Manavitehrani, I., et al., Polymers 2016, 8 (1).
2. Teo, A. J. T., et al., ACS Biomater. Sci. Eng. 2016, 2 (4), 454-472.
3. U.S. Pat. No. 3,545,439.
4. Baum, M. M., et al., J. Pharm. Sci. 2012, 101 (8), 2833-2843.
5. Malcolm, R. K., et al., Antimicrob. Agents Chemother. 2012, 56 (5), 2251-2258.
6. Gunawardana, M., et al., J. Pharm. Sci. 2014, 103 (11), 3611-3620.
7. Gunawardana, M., et al., Antimicrob. Agents Chemother. 2014, 58 (4), 2262-2267.
8. Moss, J. A., et al., Microbicide Vaginal Rings. In Drug Delivery and Development of Anti-HIV Microbicides, das Neves, J.; Sarmento, B., Eds. Pan Stanford Publishing: Singapore, 2014; pp 221-290.
9. U.S. Pat. No. 8,962,010 B2.
10. Theeuwes, F., et al., Ann. Biomed. Eng. 1976, 4 (4), 343-353.
11. Headen, D. M., et al., Microsyst. Nanoeng. 2018, 4 (1), 17076.
12. Khetan, S., et al., J. Vis. Exp. 2009, (32).
13. Zhu, J., et al., Expert Rev. Med. Devices 2011, 8 (5), 607-626.
14. El-Sherbiny, I. M., et al., Glob. Cardiol. Sci. Pract. 2013, 2013 (3), 316-342.
15. Vegas, A. J., et al., Nat. Biotechnol. 2016, 34 (3), 345-352.
16. Gomez-Florit, M., et al., Molecules 2020, 25 (24).
17. Vallet-Regi, M., et al., Eur. J. Inorg. Chem. 2003, (6), 1029-1042.
18. Ahola, M., et al., Int. J. Pharm. 2000, 195 (1-2), 219-227.
19. Giri, S., et al., Nanomedicine 2007, 2 (1), 99-111.
20. Kaity, S., et al., J. Adv. Pharm. Technol. Res. 2010, 1 (3), 283-290.
21. Choi, S. J., et al., ACS Appl. Mater. Interfaces 2011, 3 (12), 4552-4556.
22. Yu, C. L., et al., Adv. Mater. Interfaces 2017, 4 (3).
23. Calcagnile, P., et al., ACS Nano 2012, 6 (6), 5413-5419.
24. Tejashri, G., et al., Acta Pharm. 2013, 63 (3), 335-358.
25. Deuber, F., et al., Chemistry Select 2016, 1 (18), 5595-5598.
26. Deuber, F., et al., ACS Appl. Mater. Interfaces 2018, 10 (10), 9069-9076.
27. Amin Yavari, S., et al., Biomaterials 2014, 35(24), 6172-6181.
28. Li, X., et al., Regen. Biomater. 2015, 2 (3), 221-228.
29. Yasenchuk, Y., et al., Materials 2019, 12 (15).
30. Heikkinen, M. S. A., et al., J. Aerosol Sci. 2000, 31 (6), 721-738.
31. Ou, Q., et al., Aerosol Sci. Technol. 2017, 51 (11), 1303-1312.
32. Uhlmann, E., et al., Procedia CIRP 2015, 35, 55-60.
33. Grummon, D. S., et al., Appl. Phys. Lett. 2003, 82 (16), 2727-2729.
34. Lindahl, C., et al., ISRN Biomater. 2013, 2013, 205601.
35. Shi, J. R., et al., J. Mat. Chem. B 2016, 4 (46), 7415-7422.
36. Si, P. X., et al., Polym. Adv. Technol. 2015, 26 (9), 1091-1096.
37. Priyadarshani, J., et al., AIP Conf. Proc. 2020, 2270, 020004.
38. Chakraborty, S., et al., Adv. Drug Deliv. Rev. 2009, 61 (12), 1043-1054.
39. Ball, C., et al., PLoS One 2012, 7 (11), e49792.
40. Blakney, A. K., et al., Antiviral Res. 2013, 100, S9-S16.
41. Ball, C., et al., Antimicrob. Agents Chemother. 2014, 58 (8), 4855-4865.
42. Krogstad, E. A., et al., Int. J. Pharm. 2014, 475 (1-2), 282-291.
43. Chou, S. F., et al., J. Control. Release 2015, 220, 584-591.
44. Ball, C., et al., Mater. Sci. Eng. C-Mater. Biol. Appl. 2016, 63, 117-124.
45. Blakney, A. K., et al., ACS Biomater. Sci. Eng. 2016, 2 (9), 1595-1607.
46. Carson, D., et al., Pharm. Res. 2016, 33 (1), 125-136.
47. Katta, P., et al., Nano Lett. 2004, 4 (11), 2215-2218.
48. Sundaray, B., et al., Appl. Phys. Lett. 2004, 84 (7), 1222-1224.
49. Dalton, P. D., et al., Polymer 2005, 46 (3), 611-614.
50. Liu, L. Q., et al., Appl. Phys. Lett. 2007, 90 (8).
51. Zheng, J., et al., Nanoscale Res. Lett. 2015, 10 (1), 475.
52. Kroschwitz, J. I., Encyclopedia of Polymer Science and Engineering. 2nd ed.; John Wiley & Sons: New York, 1986; Vol. 6.
53. U.S. Pat. No. 3,767,756.
54. Park, S. K., et al., Fibers Polym. 2000, 1 (2), 92-96.
55. Kirschman, J. L., et al., Nucleic Acids Res. 2017, 45 (12).
56. Tiwari, P. M., et al., Nat. Commun. 2018, 9.
57. Loomis, K. H., et al., Bioconjugate Chem. 2018, 29 (9), 3072-3083.
58. Cheng, G., et al., Proc. Natl. Acad. Sci. U.S.A 2008, 105 (8), 3088-3093.
59. Alexandre, K. B., et al., Virology 2012, 423 (2), 175-186.
60. Teh, A. Y. H., et al., Plant Biotechnol. J. 2014, 12 (3), 300-311.
61. Shaughnessy, J., et al., J. Immunol. 2018, 201 (9), 2700-2709.
62. Chakraborti, S., et al., Infect. Immun. 2020, 88 (2).
63. Gulati, S., et al., J. Immunol. 2020, 204 (12), 3283-3295.
64. Shaughnessy, J., et al., Front. Immunol. 2020, 11, 583305.
65. Anderson, D. J., et al., Biol. Reprod. 2020, 103 (2), 275-285.
66. Shrestha, B., et al., Acta Biomater. 2020, 117, 226-234.
67. Oh, J. E., et al., Mucosal Vaccines for Genital Herpes. In Innovation for Preventing Infectious Diseases, 2nd. ed.; Kiyono, H.; Pascual, D. W., Eds. Adademic Press: Cambridge, MA, 2020; pp 723-734.
68. U.S. Pat. No. 5,964,804.
69. Fine, D., et al., Lab bernaChip 2010, 10 (22), 3074-3083.
70. Bernards, D. A., et al., Nano Lett. 2012, 12 (10), 5355-5361.
71. Madou, M. J., Fundamentals of Microfabrication and Nanotechnology. 3rd ed.; CRC
Press: Boca Raton, F L, 2011; p 1992.
72. Clarke, R. A., et al. Closed Porous Chambers for Implanting Tissue in a Host.
U.S. Pat. No. 5,344,454, Sep. 6, 1994.
73. U.S. Pat. No. 6,060,640.
74. Lathuiliere, A., et al., Brain 2016, 139, 1587-1604.
75. Leach, M. K., et al., J. Vis. Exp. 2011, 47, e2494.
76. Wu, Y. H., et al., E-Polymers 2017, 17 (1), 39-44.
77. Huang, Z. X., et al., Mater. Manuf. Process. 2018, 33 (2), 202-219.
78. Jiang, H. L., et al., J. Control. Release 2014, 193, 296-303.
79. Yu, D. G., et al., Chem. Commun. 2017, 53 (33), 4542-4545.
80. Yoo, H. S., et al., Adv. Drug Deliv. Rev. 2009, 61 (12), 1033-1042.
81. Quiros, J., et al., Polymer Reviews 2016, 56 (4), 631-667.
82. Badrossamay, M. R., et al., Nano Lett. 2010, 10 (6), 2257-2261.
83. Mellado, P., et al., Appl. Phys. Lett. 2011, 99 (20), 203107.
84. Wang, L., et al., Microelectron. Eng. 2011, 88 (8), 1718-1721.
85. Gonzalez, G. M., et al., Macromot Mater. Eng. 2017, 302 (1), 1600365.
86. Doyle, M. P., et al., J. Biol. Chem. 1999, 274 (4), 2583-2591.
87. Svergun, D. I., et al., Biophys. J. 2008, 94 (1), 173-181.
88. Luo, Z., et al., Sci. Rep. 2016, 6 (1), 23393.
89. Yang, J., et al., Biomaterials 2018, 182, 145-156.
90. Li, X., et al., Front. Mol. Biosci. 2021, 8 (604).
91. Li, N., et al., J. Biomed. NanotechnoL 2018, 14 (12), 2162-2171.
92. Sun, Y., et al., Acta Pharm. Sin. 2020, 10 (8), 1382-1396.
93. Ren, H., et al., ACS Appl. Mater. Interfaces 2017, 9 (4), 3463-3473.
94. Jia, X., et al., ACS Appl. Mater. Interfaces 2015, 7(8), 4579-4588.
95. Chan, W. M., et al., Clin. Exp. Ophthalmol. 2005, 33 (6), 611-618.
96. Iijima, M., et al., Int. J. Oncol. 2018, 52 (3), 679-686.
97. Owen, J., et al., PLoS ONE 2016, 11 (12), e0168088.
98. Song, R., et al., ACS Appl. Mater. Interfaces 2018, 10 (43), 36805-36813.
99. Cheng, H., et al., Adv. Funct. Mater. 2016, 26 (43), 7847-7860.
100. Zou, M. Z., et al., Small (Weinheim an der Bergstrasse, Germany) 2018, 14 (28), e1801120.
101. Phua, S. Z. F., et al., ACS Nano 2019, 13 (4), 4742-4751.
102. Li, G., et al., Adv. Funct. Mater. 2019, 29 (40), 1901932.
103. Meng, Z., et al., Adv. Mater. 2019, 31 (24), e1900927.
104. Li, G., et al., ACS Nano 2020, 14 (2), 1586-1599.
105. Song, G., et al., Adv. Mater. 2016, 28 (33), 7143-7148.
106. Wu, J., et al., Chem. Soc. Rev. 2019, 48 (4), 1004-1076.
107. Yang, G., et al., Nat. Commun. 2017, 8 (1), 902.
108. Song, X., et al., Nano Lett. 2018, 18 (10), 6360-6368.
109. Wang, J., et al., Micro Nano Lett. 2019, 11 (1), 74.
110. Escale, P., et al., Eur. Polym. J. 2012, 48 (6), 1001-1025.
111. Zhu, N., et al., Biofabrication of Tissue Scaffolds. In Advances in Biomaterials Science and Biomedical Applications, Pignatello, R., Ed. InTech: Rijeka, Croatia, 2013; pp 315-328.
112. Brazinsky, I., et al. Process for Preparing a Microporous Polymer Film. U.S. Pat. No. 4,138,459 A, Feb. 6, 2979.
113. Oh, I.-H., et al., Scr. Mater. 2003, 49, 1197-1202.
114. Jonathan, G., et al., Int. J. Pharm. 2016, 499 (1-2), 376-394.
115. Liaw, C. Y., et al., Biofabrication 2017, 9 (2).
116. U.S. Pat. No. 5,648,450.
117. U.S. Pat. No. 9,889,604.
118. Kozlowski, P. A., et al., Infect. Immun. 1997, 65(4), 1387-1394.
119. de Las Vecillas Sanchez, L., et al., Int. J. Mol. Sci. 2017, 18 (6), E1316.
120. Aebischer, P., et al., Nat. Med. 1996, 2 (6), 696-699.
121. Sommer, B., et al., Mol. Ther. 2002, 6 (2), 155-161.
122. Bensadoun, J. C., et al., J. Control. Release 2003, 87 (1-3), 107-115.
123. O'Hanlon, D. E., et al., PLoS One 2013, 8 (11), e80074.
124. Chang, T. L. Y., et al., Proc. Natl. Acad. Sci. U.S.A 2003, 100 (20), 11672-11677.
125. Kwant, A., et al., Vaccine 2004, 22 (23-24), 3098-3104.

What is claimed is:

1. A drug delivery device, comprising a scaffold comprising one or more biocompatible materials, one or more chambers containing a plurality of cells, one or more membranes, and one or more nutrient supplementation systems.

2. The drug delivery device of claim 1, wherein the drug delivery device is adapted for intravaginal use.

3. The drug delivery device of claim 1 or 2, wherein one or more biocompatible materials are non-resorbable.

4. The drug delivery device of claim 1 or 2, wherein one or more biocompatible materials are resorbable.

5. The drug delivery device of any one of claims 1 to 4, wherein the one or more biocompatible materials comprise one or more thermoplastic polymers, one or more elastomers, one or more biocompatible metals, or combinations thereof.

6. The drug delivery device of claim 5, wherein one or more biocompatible materials comprise silicone, polyurethane, poly(ethylene-co-vinyl acetate) (EVA), or a combination thereof.

7. The drug delivery device of any one of claims 1 to 6, comprising one chamber containing a plurality of cells.

8. The drug delivery device of any one of claims 1 to 6, comprising more than one chamber containing a plurality of cells.

9. The drug delivery device of claim 7 or 8, wherein the chamber or chambers are fully enclosed.

10. The drug delivery device of any one of claims 7 to 9, wherein the chamber or chambers comprise a plurality of cells of the same type.

11. The drug delivery device of any one of claims 7 to 9, wherein the chamber or chambers comprise a plurality of cells of different types.

12. The drug delivery device of claim 11, wherein the chamber or chambers comprise a plurality of cells of two or more types.

13. The drug delivery device of claim 11 or 12, wherein the chamber or chambers comprise a plurality of cells of two types.

14. The drug delivery device of claim 11 or 12, wherein the chamber or chambers comprise a plurality of cells of three types.

15. The drug delivery device of any one of claims 1 to 14, wherein the plurality of cells comprises bacterial cells, fungal cells, mammalian cells, or a combination thereof.

16. The drug delivery device of claim 15, wherein the plurality of cells comprises bacterial cells.

17. The drug delivery device of claim 15 or 16, wherein the bacterial cells comprise one or more members of the Lactobacillus genus.

18. The drug delivery device of any one of claims 15 to 17, wherein the bacterial cells comprise Lactobacillus crispatus cells, L. gasseri cells, L. jensenii cells, L. rhamnosus cells, L. iners cells, or a combination thereof.

19. The drug delivery device of claim 18, wherein the bacterial cells comprise Lactobacillus crispatus cells.

20. The drug delivery device of claim 18, wherein the bacterial cells comprise L. jensenii cells.

21. The drug delivery device of any one of claims 16 to 20, wherein the bacterial cells comprise one type of bacterial cell.

22. The drug delivery device of any one of claims 16 to 20, wherein the bacterial cells comprise a combination of bacterial cells.

23. The drug delivery device of claim 15, wherein the plurality of cells comprises fungal cells.

24. The drug delivery device of claim 23, wherein the plurality of cells comprises Candida albicans cells.

25. The drug delivery device of claim 23 or 24, wherein the plurality of cells comprises Candida albicans cells of vaginal origin.

26. The drug delivery device of claim 15, wherein the plurality of cells comprises mammalian cells.

27. The drug delivery device of claim 26, wherein the mammalian cells comprise non-human mammalian cells.

28. The drug delivery device of claim 27, wherein the mammalian cells comprise mouse microglial cells, mouse myoblast cells, Chinese hamster ovary cells, or a combination thereof.

29. The drug delivery device of claim 26, wherein the mammalian cells comprise human cells.

30. The drug delivery device of claim 29, wherein the human cells comprise human embryonic kidney cells, cervicovaginal epithelial cells, THP-1 monocyte cells, or a combination thereof.

31. The drug delivery device of any one of claims 15 to 30, wherein the plurality of cells comprises wild type cells.

32. The drug delivery device of any one of claims 15 to 31, wherein the plurality of cells comprises recombinant cells.

33. The drug delivery device of claim 32, wherein the plurality of cells comprises C8-B4 mouse microglial cells, recombinant C2C12 cells, recombinant Chinese hamster ovary cells, recombinant human embryonic kidney 293 (HEK-293) cells, 293LTV cells, recombinant cervicovaginal epithelial cells, recombinant THP-1 monocyte cells, or a combination thereof.

34. The drug delivery device of any one of claims 1 to 33, wherein the plurality of cells is enclosed within or disposed on a biocompatible matrix material within the chamber.

35. The drug delivery device of claim 34, wherein the biocompatible matrix material comprises a hydrogel.

36. The drug delivery device of claim 35, wherein the hydrogel comprises PVA, sodium alginate, hyaluronic acid, PLGA-co-PEG, biomimetic poly(ethylene glycol) gel, or a combination thereof.

37. The drug delivery device of claim 36, wherein the hydrogel comprises PVA-sodium alginate blend, hyaluronic acid-PLGA-co-PEG, or biomimetic poly(ethylene glycol) gel.

38. The drug delivery device of any one of claims 1 to 37, wherein the plurality of cells produce one or more agents.

39. The drug delivery device of claim 38, wherein the one or more agents comprise vaccines, peptides, proteins, nucleic acids, or small molecules.

40. The drug delivery device of claim 39, wherein the one or more agents comprise one or more small molecules.

41. The drug delivery device of claim 40, wherein the one or more small molecules comprise lactic acid.

42. The drug delivery device of claim 39, wherein the one or more agents comprise one or more proteins.

43. The drug delivery device of claim 42, wherein the one or more proteins comprise an antiviral protein.

44. The drug delivery device of claim 42 or 43, wherein the one or more proteins comprise one or more antibodies, antibody fragments, or nanobodies.

45. The drug delivery device of claim 44, wherein the one or more antibodies comprise a nonhormonal contraceptive comprising immunoglobulin G (IgGs) which trap sperm.

46. The drug delivery device of claim 45, wherein the one or more agents comprise a neutralizing antibody.

47. The drug delivery device of claim 46, wherein the neutralizing antibody is an HIV-neutralizing antibody.

48. The drug delivery device of claim 43, wherein the antiviral protein comprises a lectin.

49. The drug delivery device of claim 39, wherein the one or more agents comprise one or more peptides.

50. The drug delivery device of claim 49, wherein the one or more peptides comprises an antiviral peptide, an anti-cancer peptide, a messenger RNA (mRNA), or combinations thereof.

51. The drug delivery device of claim 39, wherein the plurality of cells produces one or more phages.

52. The drug delivery device of any one of claims 1 to 51, comprising one membrane.

53. The drug delivery device of any one of claims 1 to 51, comprising more than one membrane.

54. The drug delivery device of claim 52 or 53, wherein the membrane or membranes comprise a biocompatible material.

55. The drug delivery device of any one of claims 52 to 54, wherein the membrane or membranes are semipermeable.

56. The drug delivery device of any one of claims 52 to 55, wherein the membrane or membranes comprise polyester, polypropylene, polycarbonate, polyethylene terephthalate (PET), anisotropic materials, polysulfone (PSF), microfiber or nanofiber mats, polyimide, tetrafluoroethylene/polytetrafluoroethylene (PTFE), expanded polytetrafluoroethylene (ePTFE), poly(ethylene-co-vinyl acetate) (EVA), polyacrylonitrile, polyethersulfone, acrylic resin, cellulose acetate, cellulose nitrate, polyamide, hydroxylpropyl methyl cellulose (HPMC), or a combination thereof.

57. The drug delivery device of any one of claims 52 to 56, wherein the membrane or membranes comprise ePTFE, PTFE, polypropylene, poly(ethylene-co-vinyl acetate) (EVA), cellulose acetate, or a combination thereof.

58. The drug delivery device of claim 57, wherein the membrane or membranes comprise ePTFE.

59. The drug delivery device of any one of claims 52 to 58, wherein the membrane or membranes comprise a laminated structure.

60. The drug delivery device of claim 59, wherein the membrane or membranes comprise a mesh layer.

61. The drug delivery device of any one of claims 1 to 60, wherein the one or more nutrient supplementation systems comprise nutrients, growth factors, hormones, vitamins, O₂-generating agents, pH buffering agents, cell culture media, antibiotics, or a combination thereof.

62. The drug delivery device of claim 61, wherein the one or more nutrient supplementation systems comprise glucose, glycogen, peptone, amino acids, proteins, antibiotics, or a combination thereof.

63. The drug delivery device of claim 62, wherein the one or more nutrient supplementation systems comprise D-glucose-glycogen, 2:1 w/w; or D-glucose-peptone, 2:1 w/w.

64. The drug delivery device of claim 62, wherein the one or more nutrient supplementation systems comprise RPMI 1640, L-glutamine, HEPES, heat-inactivated fetal bovine serum, Normocin™, and Pen-Strep.

65. The drug delivery device of any one of claims 61 to 64, wherein the one or more nutrient supplementation systems comprise a time-release additive.

66. The drug delivery device of claim 65, wherein the time-release additive comprises PLGA-coated beads.

67. The drug delivery device of any one of claims 1 to 66, wherein the drug delivery device is an intravaginal ring (IVR), an intrauterine device (IUD), or a pessary.

68. The drug delivery device of claim 67, wherein the drug delivery device is an intravaginal ring (IVR).

69. The drug delivery device of any one of claims 1 to 68, which is physically stable at about 0-50° C.

70. The drug delivery device of claim 69, which is physically stable at about 30-40° C.

71. The drug delivery device of claim 69 or 70, which is physically stable at about 37° C.

72. A method of treating or preventing a disease or disorder in a subject in need thereof, comprising administering to the subject the drug delivery device of any one of claims 1 to 71.

73. The method of claim 72, wherein the disease or disorder is a sexually transmitted infection (STI).

74. The method of claim 73, wherein the sexually transmitted infection is Neisseria gonorrhea, Chlamydia trachomatis, Herpes Simplex Virus, Treponema pallidum, or Human Immunodeficiency Virus (HIV) infection.

75. A method of contraception, comprising administering to a subject in need thereof the drug delivery device of any one of claims 1 to 71.
